(19) United States
(12) Patent Application Publication
(10) Pub. No.: US 2026/0354830 A1
(43) Pub. Date: Nov. 12, 2026

(54) GASTROENTEROANASTOMOSIS DEVICE

(71) Applicant: GEAbetes ApS, Struer (DK)

(72) Inventors: Einar Gjaldbaek PETERSEN, Struer (DK); Ole KJELDSEN, Struer (DK); Henrik HARBOE, Struer (DK); Tue Kjaergaard TOFT, Struer (DK); Peter VILMANN, Struer (DK); Adrian SAFTOIU, Struer (DK)

(73) Assignee: GEAbetes ApS, Struer (DK)

(21) Appl. No.: 19/139,323

(22) PCT Filed: Dec. 18, 2023
(86) PCT No.: PCT/EP2023/086290

§ 371 Date: Jun. 13, 2025

(30) Foreign Application Priority Data
Dec. 16, 2022 (DK) PA 2022 70623

(51) Int. Cl. A61B 17/11; A61B 17/00

(52) U.S. Cl. CPC A61B 17/1114; A61B 2017/00022; A61B 2017/00818; A61B 2017/1139

(57) ABSTRACT

A gastroenteroanastomosis device for use in a minimally invasive procedure for forming a passage across apposed tissue walls from the stomach to a target of the small intestines. The device transforms from a collapsed state for minimally invasive delivery to a gastro-enteric deployment site to a deployed state for deployment at the gastro-enteric deployment site. The device features: a first part for enteric placement; a second part for gastric placement; and a centrally arranged mechanical connection forcing the parts towards each other. First and second engagement regions of the parts engage the apposed tissue walls therebetween to apply an ischemic pressure. First and second peripheral regions of the parts contact the apposed tissue walls therebetween to apply a non-ischemic pressure. The first and second peripheral regions gradually diverge from each other and cooperate to gradually reduce a pressure applied to the tissue walls from ischemic pressure at the engagement region to no pressure at the periphery.

The present invention relates in one aspect to an anastomosis device for creating a passage across two tissue walls defining two body lumens for use in endoscopic procedures. In a particularly advantageous aspect, an anastomosis device for use in endoscopic gastrointestinal procedures is provided for creating a passage connecting a first lumen delineated by a first tissue wall and a second lumen delineated by a second tissue wall. More particularly, the invention relates to a device for endoscopic gastroenteroanastomosis adapted for delivery in an endoscopic procedure through a natural orifice. Furthermore, the invention relates to a gastroenteroanastomosis device for use in a gastric bypass procedure connecting the stomach with a portion of the small intestines while at the same time blocking, reducing, or actively controlling flow from the stomach through the pylorus to the duodenum by means of a pylorus plug, thereby at least partially bypassing at least a portion of the duodenum.

### BACKGROUND OF THE INVENTION

Obesity is at the root cause of many diseases. Comorbidities of obesity may include pulmonary and cardiovascular diseases, arthritis, and type-2 diabetes. A successful treatment addressing obesity is gastric bypass surgery, creating a gastric pouch reducing the active stomach volume, and bypassing the remaining stomach volume and the duodenum, thereby significantly reducing a patient's uptake of nutrients.

Such surgical interventions are costly and involve many risks and complications. Recent research efforts therefore target developing minimally invasive procedures for bypassing at least a portion of the duodenum, which may at least partially be performed endoscopically or laparoscopically.

US 2013/0325042 A1 describes an endoscopic method, which comprises creating an anastomosis between a stomach and a portion of a small intestine, and simultaneously controlling passage of stomach contents through a pylorus with a pylorus plug that comprises a valve operative to either close or at least partially open passageway through the pylorus. According to US 2013/0325042 A1, the anastomosis is created using a magnetic anastomosis device.

A review article in the Journal of Gastrointestinal Endoscopy, Volume 93, No. 1, pp.34-46 reviews a variety of minimally invasive techniques used for endoscopic gastrointestinal anastomosis procedures. The techniques discussed in this article include, among others, endoscopic ultrasound guided gastroenterostomy using stents, anastomosis formation using magnetic devices, and compression devices. However, referring to the underlying studies reporting the different techniques, numerous challenges and complications of the known techniques are also described in this review article.

Complications encountered in gastrointestinal anastomosis include infections resulting from leakage of gastric fluids into the abdominal cavity, or the entrapment of vis-cera during placement of an anastomosis device. A particular source of complications are magnetic devices that may be difficult to deliver and place in a reliable manner, or may require elaborate and costly monitoring equipment.

Challenges include complicated procedures involving multiple surgical techniques, thereby increasing the risk for complications to occur. Challenges may also include that certain techniques require prolonged interventions or multiple subsequent interventions, or that certain procedures in daily practice may be difficult to perform in a reliable and reproducible manner. Other issues may include the need for replacing implanted devices regularly, such as stents requiring annual interventions for re-placement. Yet further challenges are inherent to the use of magnetic devices, which may cause interference with other equipment used during the surgical procedures, such as endoscopes or surgical instruments including magnetic materials, or interference with defibrillators.

U.S. Pat. No. 11,076,856 describes an anastomosis device configured to reduce leakage. The device comprises a first set of rings sized to be positioned at a first tissue wall, a second set of rings sized to be positioned at a second tissue wall adjacent the first tissue wall and an element configured to draw the ring sets towards each other.

Each set of rings comprises an inner ring, an outer ring, and a coupling for aligning the inner and outer rings with respect to each other. The inner ring applies a first pressure onto the tissue wall, which is sufficient to gradually form a passage between the first and second tissue walls. The outer ring applies a second pressure onto the tissue wall radially outwardly relative to the first pressure, which is sufficient to reduce leakage in a vicinity of the passage. However, the device disclosed in U.S. Pat. No. 11,076,856 is complicated, requiring the multiple rings to be delivered and correctly placed. The disclosed device further requires adjustment and control of the different pressures applied to the tissue entrapped by the inner and outer rings of the first and second ring sets. Furthermore, U.S. Pat No. 11,076,856 strongly suggests controlling or adjustment of the different applied pressures over time to achieve the desired effect, i.e. a reliable formation of the anastomosis with reduced leakage.

Therefore, there is a need for a gastroenteroanastomosis technique overcoming at least some of the problems of known techniques or providing an alternative. In particular, there is a need for devices for use in such gastroenteroanastomosis procedures, which are adapted for endoscopic delivery, in particular for delivery through an instrument channel of an endoscope for entry through natural orifices, and which are suitable for use in a gastric bypass procedure.

### SUMMARY OF THE INVENTION

Object of the present invention is to provide a device and a system for endoscopic gastroenteroanastomosis overcoming at least some of the problems of known techniques or providing an alternative. A particular object is to provide a device or a system for use in a simplified gastric bypass procedure for forming a passage from the stomach to the small intestines bypassing the pylorus and at least a portion of the duodenum. A further particular object of the invention is to eliminate the use of magnets applying pressure to entrapped tissue walls for forming such a passage. A further object is to eliminate permanent implants for providing a passage from the stomach to the small intestines bypassing the pylorus and at least a portion of the duodenum. A further object is to provide such a device or system for use in endoscopic procedures, and which provides a high level of patient safety. A yet further object is to provide such a device or system facilitating reliable placement and easy alignment. A yet further object is to provide such a device or system for use in a minimally invasive gastric bypass procedure reducing duration of the intervention to be performed on a patient, and/or reducing the number of interventions to be performed on the patient. A yet further object is to provide such a device or system for use in a minimally invasive gastric bypass procedure that may be reversed in a simple manner.

One or more of these objects of the invention are achieved by a device as defined in the attached independent claim(s) with advantageous embodiments as defined by the dependent claims and as disclosed herein. One or more of these objects of the invention are furthermore achieved by a system comprising such a device as defined in the attached independent claim(s) with advantageous embodiments as defined by the dependent claims and as disclosed herein.

The gastroenteroanastomosis device according to embodiments of the invention is useful for use in a gastric bypass procedure connecting the stomach with a portion of the small intestines while at the same time blocking, reducing, or actively controlling flow from the stomach through the pylorus to the duodenum by means of a pylorus plug, thereby at least partially bypassing at least a portion of the duodenum. Advantages of the invention are described in the following by referring to the embodiments as disclosed herein, throughout the entire disclosure.

The term “anastomosis” as used herein refers to healing apposed tissue walls together along a circumferentially closed path providing a peripheral seal for a passage formed in the process. The passage is formed and opened by causing necrosis within the portion of the tissue walls enclosed by the circumferentially closed peripheral seal. Necrosis is caused by applying an ischemic pressure to tissue engaged within the region of the passage, inward of the peripheral seal, so as to suppress blood flow into said region. Healing together of the apposed tissue walls in the portions of the apposed tissue walls forming the peripheral seal is achieved by keeping the tissue walls in contact with each other under non-ischemic compression allowing for proper blood flow in the seal forming portions. The device according to embodiments of the invention is deployed across apposed portions of gastric and enteric tissue walls. The terms “gastro” and “gastric” as used herein refer to the stomach, and the terms “entero” and “enteric” as used herein refer to the small intestines. More specifically, the gastroenteroanastomosis device according to embodiments of the invention is conceived for deployment across apposed portions of the gastric body wall and of a target region of the enteric tube wall. The terms “proximal” and “distal” as used herein when denoting parts of the device and instrumentation for its minimally invasive deployment respectively refer to directions towards, or locations close to, an operator end thereof (proximal) and directions pointing away from, or locations distant from, an operator end thereof, as common in the art. The terms “upstream” and “downstream” as used herein when referring to portions of the gastrointestinal tract are to be understood with reference to the normal flow of gastric matter from the mouth, through the stomach and the bowels towards the rectum. The pylorus defines the passageway between the stomach and the duodenum and includes a pyloric sphincter for controlling the passage of stomach contents into the intestines. The term “pylorus” as used herein generally refers to the area of the opening from the stomach to the duodenum, and includes locations both upstream of the pyloric sphincter and downstream of the pyloric sphincter. “Axial directions” generally refer to directions from a proximal to a distal end of the device. With respect to endoscopes, endoscopic instrument channels, guidewires, catheters, or similar channels and instruments for minimally invasive procedures, the term “axial” denotes directions along a principal axis thereof, following the direction of introduction into the patient. When considering the device according to embodiments of the invention, with reference to its deployment across apposed tissue walls, the term axial denotes directions essentially perpendicular to the apposed tissue walls portions, parallel to its direction of insertion and penetration through the apposed tissue wall portions. Transverse directions are perpendicular to the axial direction, such as radial directions. When considering the device according to embodiments of the invention, with reference to its deployment across apposed tissue wall portions, two particular transverse directions may be identified as “longitudinal”, for directions along a generally lengthwise direction of the intestinal tube as seen at the target region, and cross-wise, for directions from side to side of the intestinal tube as seen at the target region, i.e. perpendicular to both the axial and longitudinal directions. The terms “central”, “circumferential”, “outward”, “peripheral”, or “peripherally outward” are understood as seen in transverse directions. The term “central” means typically close to the mechanical connection connecting first and second parts of the gastroenteroanastomosis device with each other, or comparatively closer to the mechanical connection as compared to a location on the device described as outward, peripheral, or peripherally outward. In a gastroenteroanastomosis device as disclosed herein, an “outward” direction thus points in a transverse direction, typically radially, away from a centrally located mechanical connection defining a central axis A. Correspondingly, an “inward” direction points in a transverse direction, typically radially, towards the centrally located mechanical connection defining a central axis A.

A first aspect of the invention relates to a gastroenteroanastomosis device for use in a minimally invasive procedure for forming a passage across apposed tissue walls from the stomach to a target portion of the small intestines in a patient; the device being transformable from a collapsed state configured for minimally invasive delivery of the device to a gastro-enteric deployment site in the apposed tissue walls to a deployed state configured for deployment at the gastro-enteric deployment site; the device comprising, when seen in the deployed state: a first part adapted for enteric placement; a second part adapted for gastric placement; and a centrally arranged connection forcing the first and second parts towards each other in an axial direction; wherein the first part comprises a first engagement region, wherein the second part comprises a second engagement region facing towards the first engagement region, and wherein the first and second engagement regions are adapted to engage the apposed tissue walls there between to apply an ischemic pressure; wherein the first part further comprises a first peripheral region enclosing the first engagement region in a peripherally outward direction, wherein the second part further comprises a second peripheral region enclosing the second engagement region in a peripherally outward direction and facing towards the first peripheral portion, and wherein the first and second peripheral regions are adapted to contact the apposed tissue walls there between to apply a non-ischemic pressure; wherein the first and second peripheral regions gradually diverge from each other, whereby the first and second peripheral regions cooperate to gradually reduce, in an outward going direction from the engagement region to a periphery of the device, a pressure applied to the apposed tissue walls from ischemic pressure at the engagement region to no pressure at the periphery, passing through non-ischemic pressure.

The connection is a mechanical connection. The connection is attached to center regions of the first and second parts. The connection penetrates the apposed tissue walls in a center portion thereof, forcing (by pulling and/or pushing) the first and second parts towards each other. The first and second parts comprise cooperating surfaces adapted to hold the apposed tissue walls there between. The cooperating surfaces of the first and second parts comprise the respective cooperating engagement and peripheral regions. The cooperating parts are thus adapted to apply ischemic pressure throughout the engagement regions, and the cooperating parts are further adapted to apply non-ischemic pressure to the apposed tissue walls in the peripheral regions. The first and second peripheral regions gradually diverge from each other, to gradually increase the distance between the surfaces from an ischemic engagement distance at the engagement region to a distance equal to or exceeding the sum of the uncompressed thickness of the apposed tissue walls, where no pressure is applied. The apposed tissue wall portions are only penetrated for the enteric placement of the first part, and only with a perforation having a very small circumference corresponding to the perimeter of the first part in its collapsed state, as seen in transverse directions. The first and second engagement regions enclose (or comprise) the respective center regions of the first and second parts. The peripheral regions fully enclose the corresponding engagement regions, and thus also the corresponding center portions where the penetration is placed, thereby providing a reliable and circumferentially complete seal already upon deployment of the device, once the second part is seated against the first part across the apposed tissue walls. Consequently a risk of gastric leakage, if any, is reduced to a short circumferential length of the first penetration aperture, and to a short period in time during an intervention, where the patient is prepared with infection suppressing medicine.

Most preferably, the device according to embodiments of the invention is configured to prevent transport of gastric matter through the passage during formation of the passage, i.e. the passage is not open while the device is deployed at the site of passage formation. In other words, the devices of the present disclosure are not conceived to provide a functional transluminal shunt from the stomach to the small intestines while they are installed. The actual passage is only opened at the end of the procedure when removing the device again, i.e. after the gastroenteroanastomosis providing the permanent peripheral seal is fully healed. This further minimizes risk of gastric/enteric leakage into the abdominal cavity during the formation of the gastroenteroanastomosis passage, i.e. throughout the period of device deployment.

By requiring gradually diverging peripheral regions a smooth transition is achieved, where compression of the apposed tissue walls always goes from an ischemic regime causing necrosis in the engagement regions to no compression at the outer periphery where the apposed tissue walls are fully released, passing through a non-ischemic regime throughout the peripheral regions, where the apposed tissue walls heal together to reliably form a permanent peripheral seal. The practitioner can thus focus on deploying the device for correct compression in an ischemic regime. The gradually diverging peripheral regions that circumferentially enclose the ischemic engagement regions then ensure a proper seal already upon deployment, and a proper formation of a permanent seal by anastomosis.

In the deployed state, the second part is thus seated against the first part, the two parts engaging the two apposed tissue walls between them. When installing the device, the practitioner (i.e. the operator or user of the device/endoscopic system) adjusts the mutual position of the first and second parts according to a desired pressure distribution applied to the engaged tissue walls by adjusting the tension of the mechanical connection between the first part and the second part. Advantageously in some embodiments, the device and/or an endoscopic system for the delivery and placement of the device comprises a sensor element that provides an output indicative of an axial tension applied to pull and/or push the first and second parts towards each other. Collecting this output by a wired connection or by wireless communication allows to monitor and/or document the proper tensioning of the device during installation, and if the sensor element is arranged in the device itself, even during the period of placement of the device in a patient for the formation of a gastroenteroanastomosis passage. For example, the sensor element may be adapted to communicate with an indicator device arranged at a proximal end of the endoscope. The indicator device may then provide user-perceptible output to the operator/user, thus allowing the operator/user to monitor and document the correct placement and tension of the device. Furthermore, the indicator device, or a further data processing device may also collect the output from the sensor element for the purpose of monitoring, logging, and documenting correct placement and/or proper tensioning of the device during and/or after the installation, and/or even during the entire indwelling time of the device. The indicator device and/or the further data processing device may further comprise programmed instructions allowing for checking the collected sensor element output against pre-determined values for proper tensioning. For example, such predetermined values may be given and/or stored in a memory associated with the indicator device or further processing device as threshold values, upper and/or lower limits defining corresponding ranges for proper tensioning, or the like. Furthermore, the indicator device and/or the further processing device may be configured to compare the sensor element output with the predetermined values for proper tensioning, and provide a user-perceptible warning and/or alarm, if the sensor output approaches and/or exceeds (falls outside) the predetermined values for proper tensioning. Once the two parts are installed with proper tension forcing (by pulling and/or pushing) the first and second parts towards each other, their placement with respect to each other is typically fixed until the device eventually is removed in a second intervention, after formation of the gastroenteroanastomosis passage, as described herein.

The gradual transition is configured according to a non-ischemic region having a pre-determined minimum width, even though an exact wall thickness may not be known for the individual in question. To that end the device may be designed according to have regions that gradually diverge over a width corresponding to, or exceeding, a desired seam width of the anastomotic seal to be formed. Advantageously, the device may be dimensioned according to typical average values in a subject without gastric or enteric wall thickness abnormalities. However, by requiring a gradual transition over a predetermined width corresponding to, or exceeding, a desired seam width as seen in the plane of the apposed tissue walls, the device is largely insensitive, or at least less sensitive to variations in gastric and enteric wall thicknesses than known devices.

Furthermore, the gradual transition avoids any stepwise transitions, which may cause complications, such as cutting holes, severing the clamped tissue by shearing under the influence of gastric and/or enteric activity in the period of time where the device is deployed. The gradual transition also avoids jumping of the compression pressure from an ischemic regime to insufficiently low pressures jeopardizing the application of a proper non-ischemic contact pressure required to provide a proper seal upon deployment, and to form a properly healed permanent seal around the passage to be formed. By requiring a gradual divergence of cooperating peripheral regions surrounding the ischemic engagement regions the gastroenteroanastomosis device according to embodiments of the invention becomes less sensitive to variations in tissue wall thicknesses from patient to patient, and has an increased robustness against certain variations, e.g. from intervention to intervention, in the correct deployment of the device, such as regarding precision of the actual force transmitted by the connection to force the first and second parts towards each other.

Further according to some embodiments of the device, the distance between the cooperating peripheral regions increases monotonically, or strictly monotonically, from an inner distance at the peripherally outer border of the engagement regions to an outer distance at the peripherally outer border of the peripheral regions.

Advantageously within the peripheral regions, the distance between the cooperating first and second peripheral regions on the cooperating surfaces of the first and second parts increases monotonously, or strictly monotonously, from an engagement distance, at the peripheral contour (perimeter) of the engagement regions, to a release distance corresponding to the sum of the thicknesses of the uncompressed apposed tissue walls, at the outer periphery of the peripheral regions.

Requiring monotonously, or even strictly monotonously, diverging peripheral regions further enhances the above-mentioned advantages of a smooth transition from ischemic engagement to no pressure at the periphery, for providing a reliable peripheral seal. By requiring at least monotonously diverging peripheral regions it is ensured that the a non-ischemic compression regime is maintained throughout the peripheral regions without returning to an ischemic regime, and without releasing the apposed tissue between the outer contour of the engagement regions and the outer periphery of the peripheral regions. The device thus becomes even less sensitive to variations in tissue wall thicknesses from patient to patient, and robustness against certain variations in the deployment of the device is even further improved.

Further according to some embodiments of the device, a first one of the first and second cooperating parts has a concave shape facing towards the other one of the first and second parts.

By providing a concave shape, the first one of the first and second cooperating parts is adapted to receive the other one of the first and second parts therein, thereby facilitating self-alignment of the two parts of the gastroenteroanastomosis device with respect to each other, both for the correct installation of the device and for maintaining correct alignment throughout the deployment period.

Advantageously according to some embodiments, the first one of the first and second cooperating parts has a concave shape at least as seen in a transverse cut-plane parallel to the axial direction. Further advantageously, the first one of the first and second cooperating parts has a concave shape at least as seen in two transverse cut planes parallel to the axial direction and intersecting each other. These embodiments confer improved transverse self-alignment in at least one, preferably two different transverse directions.

Advantageously according to some embodiments, the first one of the first and second cooperating parts has a concave shape at least as seen in a cross-wise cut plane parallel to the axial direction and/or in a longitudinal cut plane parallel to the axial direction. These embodiments have the advantage of conferring improved transverse self-alignment in the cross-wise and/or the longitudinal directions.

By combining the above-mentioned diverging configuration of the first and second peripheral regions with the above-mentioned concavely shaped first or second parts adapted for receiving the other one of the first and second parts, the self-alignment into correct placement when installing the device is further improved, as the diverging peripheral surfaces also serve to gradually guide the parts into correct placement.

Further according to some embodiments of the device, the other one of the first and second cooperating parts has a convex shape facing towards the concave first one of the first and second parts. Advantageously according to some embodiments, the other one of the first and second cooperating parts has a convex shape at least as seen as seen in the same transverse cut-plane parallel to the axial direction as the concavely shaped counterpart. Cooperating parts that are concave-convex at cooperating surfaces facing towards each other further support an improved self-alignment.

Further according to some embodiments of the device, a peripheral contour of the cooperating engagement regions of the first and second parts has a circular shape. The circular shape may be circular or approximated circular, such as forming a closed regular polygon with at least 5, at least 6, at least 7, at least 8, or more sides. Preferably, the corresponding peripheral regions enclosing the engagement regions also have a circular, or approximated circular contour geometry. By combining a circular shape with cooperating parts that are concave, or concave-convex at cooperating surfaces facing towards each other the improved self-alignment also includes improved self-centering.

Advantageously according to some embodiments of the device, a first part for enteric placement has in its deployed state a convex surface facing towards a cooperating surface of the second part, and the cooperating surface of the second part for gastric placement has in its deployed state a concave surface facing towards the convex surface of the first part. The first and second parts are connected through a centrally located mechanical connection penetrating apposed gastric and enteric walls at a target site. The mechanical connection provides an axial force that pulls and/or pushes the first and second parts towards each other, thus resulting in an axially tensioned assembly connecting the first part and the second part with an opera-tor/user-adjustable tension. Due to the combination of a convex surface with a concave cooperating surface the two parts self-align when pulled towards each other, thus facilitating an easy and reliable placement of the device. Once the mechanical connection is properly tensioned, the convex surface of the first part and the concave surface of the second part cooperate to engage the apposed tissue walls to form a circumferentially sealed anastomosis connection as discussed elsewhere herein, throughout the entire disclosure. Further advantageously, each of the first and second parts comprises a collapsible frame structure, which in a collapsed state has a transverse dimension adapted to fit in the lumen of an endoscopic catheter for endoscopic delivery and placement of the device to its target site, e.g. through the instrument channel of an endoscope. For example, the collapsible frame structure may comprise foldable legs, wire loops, and/or a mesh structure, such as a braided wire mesh. Thereby, the first and second parts of the device may be delivered and placed at the same time, i.e. in the same descent through the endoscopic channel, in a single minimally invasive intervention. Advantageously, the endoscopic intervention is performed through a natural orifice, such as entering the stomach via the mouth and esophagus.

Advantageously in some embodiments, the first part for enteric placement has in a deployed state an overall concave shape facing in a distal direction, away from the second part and into the enteric volume at the target region. Further advantageously, the first part for enteric placement comprises a collapsible frame structure, which in a deployed state forms an open cup-shape, wherein a convex side of the cup-shape faces towards the apposed tissue walls and towards a cooperating, preferably concave, surface of the second part for gastric placement, and wherein a concave opposite side of the cup-shape faces away from the apposed tissue walls and towards a lumen in the target region of the small intestines. This largely reduces the risk of obstruction of the small intestines during an indwelling period, i.e. during a period of placement of the gastroenteroanastomosis device for anastomosis formation, thereby further enhancing patient safety. As also discussed elsewhere herein, the collapsible frame structures may be made of any suitable biocompatible material.

Further according to some embodiments of the device, a peripheral contour of the cooperating engagement regions of the first and second parts has an elongated shape. An elongated shape facilitates benefits forming a passage with a relatively large perimeter s, while at the same time keeping transverse dimensions sufficiently small to facilitate the minimally invasive introduction and placement of the device. At the same time, a reliable seal is achieved in a simple manner, due to the presence of the gradually diverging peripheral surface regions, which peripherally enclose a central engagement region where the passage opening is formed by necrosis.

Advantageously, the direction of elongation is in a longitudinal direction, for deployment at a gastroenteric deployment site, the longitudinal direction being parallel to a general direction of the enteric tube at the target region. Elongation of the device may be described by a width of the device in a cross-wise direction as compared to a length of the device in a longitudinal direction, and may be expressed as an effective aspect ratio. The width is determined by the width of the cooperating parts as seen in a cross-wise direction, and the length is determined by the length of the cooperating parts as seen in a longitudinal direction. The aspect ratio may be defined as the ratio of the width to the length. An elongated shape of the device with a direction of elongation that is aligned with the longitudinal direction along the general direction of the small intestines in the target region benefits forming a passage with a relatively large perimeter s, and thus a relatively large opening as compared to the width of the tube of the small intestines. Advantageously, an aspect ratio of cross-wise to longitudinal dimensions of the device is in the range from 1:3, or 1:5, or 1:7 and up to 1:15, or up to 1:17, or up to 1:20. For example, an advantageous aspect ratio of cross-wise to longitudinal dimensions of the device is 1:8, or 1:10, or 1:12.

Advantageously according to some embodiments, an elongated shape of the engagement regions, peripherally sealed by the diverging peripheral regions, is combined with a concave shape of the first or second part for receiving the other one of the first and second parts therein, as seen in cross-wise transverse cut planes thereof. Thereby, rotational self-alignment is achieved when seating the first and second parts with their cooperating surfaces against each other to clamp the apposed tissue walls there between. Further advantageously, a cross-wise curvature of the concave shape is larger than a longitudinal curvature thereof, thereby further supporting rotational self-alignment of the first and second parts with respect to each other.

Advantageously according to some embodiments, the second part comprises an elongated trough, i.e. a concave shape facing towards the first part in the deployed state, wherein a cross-wise concave curvature is large as compared to a longitudinally concave curvature. At the limit, a trough is only curved in a cross-wise direction, whereas a longitudinal profile is essentially straight. During placement, a bar-shaped first part is received in the trough shaped second part in a self-aligning manner.

In any of the embodiments comprising a concave receiving surface, the self-aligning cooperation of the concave surface regions of one part with the corresponding surface regions of the other part is also beneficial for maintaining the correct placement of the first and second parts with respect to each other during the entire period of forming the gastroenteroanastomosis passage. The correct placement at the time of installation, and throughout the entire duration of deployment, ensures that the device applies the correct pressure distribution to the apposed tissue walls clamped between the first and second parts. Thereby, reliability and safety of the formation of the gastroenteroanastomosis passage is enhanced.

Advantageously according to some embodiments, the first part comprises an elongated bar, with a convex shape facing towards second part in the deployed state. Further advantageously, the elongated bar is tiltable about a cross-wise axis between a collapsed state, where the direction of elongation of the bar is parallel to the axial direction, and an expanded state, where the direction of elongation of the bar is perpendicular to the axial direction. Preferably, the tilt axis is located to pass through a central region of the device. Thereby, a simple construction is provided, facilitating a relatively simple production at low cost, and easy handling for introduction and placement of the device. At the same time, a reliable seal is achieved in a simple manner, due to the presence of the gradually diverging peripheral surface regions, which peripherally enclose a central engagement region where the passage opening is formed by necrosis.

Further according to some embodiments of the device, the first part and/or the second part comprises a collapsible frame structure having a collapsed state adapted for minimally invasive delivery and an expanded state adapted for deployment at the gastro-enteric deployment site. Advantageously, the collapsible frame structure comprises a wire frame, a mesh structure, and/or articulated legs. By providing a collapsible frame structure, as distinct from an inflatable structure, a collapsed state with small transverse dimensions for minimally invasive delivery is provided, which can be converted into a relatively large expanded shape at the site of deployment, without the need for much infrastructure for the activation of the expanded state, thus saving on the transverse footprint of the required instrumentation in the minimally invasive channel through which the intervention is performed. Furthermore, the actual volume taken up by a collapsible frame structure in the expanded state is also less than for an inflatable structure. This is particularly advantageous for the first part, since the first part in its expanded state is deployed in the small intestines. A small cross-sectional profile as seen in a cross-wise cut-plane, of an expanded frame structure, as compared to the spacious/large profile of an inflatable structure, reduces the risk of obstruction of the transport of gastric matter during the period of deployment of the gastroenteroanastomosis device. This enhances patient safety by reducing the risk of complications caused by such potential obstructions.

The first part may thus be configured to present, when placed in the expanded state at the enteric deployment site, a relatively small cross-sectional area as seen in a cross-wise cut-plan, as compared to a maximum cross-sectional area of the intestinal tube at the target portion.

Further according to some embodiments of the device, the first part or the second part, as applicable, further comprises a foldable membrane attached to the collapsible frame structure, wherein the membrane is folded together when the collapsible frame structure is in the collapsed state, and wherein the membrane is unfolded, or stretched out, to present a cooperating surface comprising engagement and peripheral regions, when the collapsible frame structure is in the expanded state. The first or second membrane of the first or second part defines, in its unfolded state, the engagement and peripheral regions shaped and dimensioned as discussed elsewhere herein. The cooperating surfaces of the first or second membrane are presented towards the other one of the first and second parts, as applicable. Thereby, a pressure distribution is achieved that is smoothly spread over the engagement and/or peripheral regions, while reducing the material required for spanning the required dimensions of the passage to be formed. Thereby, the transverse dimensions of the parts in the collapsed state may be kept small for a given desired span of the deployed device. Thereby, minimally invasive delivery and placement of the device is further enhanced.

Further according to some embodiments of the device, a collapsible structure of the first part or the second part is mechanically biased towards an expanded state.

Thereby, the device is adapted to automatically expand from a collapsed state to an expanded state when a transversely confining element maintaining the collapsible structure in the collapsed state is removed. This is beneficial for an easy deployment of the device.

Advantageously according to some embodiments, a collapsible structure of the first part or the second part comprises a memory material, such as nitinol, configured to transform the collapsible structure from a collapsed state to an expanded state when the memory material is activated. This is beneficial for an easy deployment of the device in a particularly well-defined and controlled manner.

Advantageously according to some embodiments, the second part comprises an inflatable element. Thereby the pressure, and the pressure distribution, applied to the tissue walls clamped between the first part and the second part may be adjusted at least at the time of delivery. Furthermore, the inflatable structure may be easily punctured for easy retrieval of the device. By requiring the second part to comprise an inflatable element, while the first part does not comprise an inflatable element, these advantages may be achieved without risking the potential complications of obstruction that may follow with the use of a relatively bulky inflatable element deployed in the small intestinal tube.

Further according to some embodiments of the device, a tip portion of the first part comprises a cutting device, such as an electrically heated cutting wire. Advantageously, the cutting device is arranged at a distal end of the first device when the first device is in a collapsed state adapted for minimally invasive delivery. This embodiment simplifies a first intervention for introducing and placing the gastroenteroanastomosis device at the steps of penetrating the apposed tissue walls at the deployment site, and subsequently deploying the first part in enteric placement.

Advantageously according to some embodiments, the cooperating engagement regions have a perimeter s of the engagement region between s=3 cm and s=12 cm; or between s=5 cm and s=10 cm; or about 7 cm.

Advantageously according to some embodiments, a width of the diverging cooperating first and second peripheral regions, as seen in a direction from a peripheral contour of the engagement regions to an outer periphery of the peripheral regions, is at least 2 mm, or at least 3 mm, or at least 4 mm, or at least 5 mm.

Advantageously according to some embodiments, a transverse/radial dimension/width of the peripheral region providing a non-ischemic pressure is at least 2 mm, or at least 3 mm, or at least 4 mm, or at least 5 mm. Requiring a minimum width of the peripheral region determines a width of the peripheral seal under a non-ischemic pressure during the period of deployment, and a sealing seam width of the permanent peripheral seal formed. Upper limits of these dimensions may be determined by spatial constraints imposed by the minimally invasive deployment and retrieval of the device. Furthermore, an upper limit is also determined by the maximum total footprint of the gastroenteroanastomosis passage in the patient and is to be balanced against the desired opening to be formed.

Advantageously according to some embodiments a distance between the cooperating engagement regions is between 0.5 mm and 2 mm, or between 0.6 mm and 1.8 mm, or between 0.7 mm and 1.4 mm, or between 0.8 mm and 1.2 mm. A minimum distance is adapted for ensuring ischemic compression of the apposed tissue walls in the engagement region, while avoiding severing the engaged tissue upon deployment. A maximum distance is adapted for ensuring ischemic compression of the apposed tissue walls in the engagement region, up to a transition to a non-ischemic compression regime. An actual peripheral contour transverse dimension of the engagement regions providing ischemic compression of the apposed tissue walls may slightly vary, depending on the actual wall thicknesses and the force applied by the axial connection forcing (pulling and/or pushing) the first and second parts towards each other, and the details of the surface geometry close to the periphery of the engagement regions.

Advantageously according to some embodiments, a the first part has a transverse diameter in the collapsed state configured for delivery through an endoscopic instrument channel, such as less than 4 mm, and/or the second part has a transverse diameter in the collapsed state configured for minimally invasive delivery to the stomach through a natural orifice, such as less than 16 mm, or less than 12 mm, or less than 10 mm, or less than 8 mm. Thereby, safe delivery of both the first part (for enteric placement) and of the second part (for gastric placement) is ensured. The two parts of the device are delivered and deployed separately. Typically, delivery and deployment of the device, and its subsequent retrieval, is performed in minimally invasive interventions, such as transorally and through the esophagus.

Advantageously according to some embodiments, a guidewire is attached to the first part, thereby being adapted for guiding minimally invasive delivery of the second part along the guidewire to the stomach.

Advantageously according to some embodiments, the second part is configured for delivery along a guidewire, such as comprising an axial channel (centrally arranged) with a lumen configured for passing a guidewire there through. For example, the lumen diameter may be between 0.5 mm-1.5mm).

Further according to some embodiments, the device further comprises a sensor element adapted to provide an output indicative of a highest compression force applied between the first part and the second part. Arranging such a compression force sensor element integrated in the gastroenteroanastomosis device for placement in the patient facilitates an in situ measurement of a compression force as applied to the apposed tissue walls, or at least a representative estimate thereof. This allows an operator/user for example to monitor the device installation and to ensure that compression of the tissue is sufficient to form a central necrosis and to provide a peripheral tissue connection such that the apposed tissue walls heal together to reliably form a permanent peripheral seal.

Further according to some embodiments, the device further comprises a sensor element adapted to provide a sensor output indicative of an axial tension forcing (pulling and/or pushing) the first part and the second part towards each other. Arranging such an axial tension force sensor element integrated in the gastroenteroanastomosis device for placement in the patient facilitates an in situ measurement of an axial tension as applied between the first part and the second part, or at least a representative estimate thereof. The axial tension pulling and/or pushing the first and second parts towards each other provides a representative measure for the highest compression force exerted by the cooperating engagement surfaces of the first and second parts. The sensor output thus gives a useful indication for assessing the correct placement with proper tensioning of the device at the deployment site. Collecting the sensor output then allows for assessing and/or documenting the quality of the placement during and/or after installation, or even throughout the period of anastomosis formation. Using the sensor output, the installation may thus be performed in a well-controlled manner. More particularly, an operator/user may use the output from the axial tension sensor element to monitor the device installation and to ensure that compression of the tissue is sufficient to form a central necrosis and to provide a peripheral tissue connection such that the apposed tissue walls heal together to reliably form a permanent peripheral seal. Furthermore, the operator can ensure that tension applied to the operating handle at the proximal end of the endoscopic instrumentation used for the installation is properly transmitted to the anastomosis device. Thereby, reproducibility of the correct placement, and ultimately patient safety is significantly enhanced.

Further according to some embodiments of the device, the sensor element is arranged in the mechanical connection between the first part and the second part. This placement of the sensor allows to collect information about the highest tension force applied to the anastomosis device. Advantageously, such a sensor element may be arranged in or at the mechanical connection between the first and second parts. Thereby, a reliable measure for the axial tension may be obtained.

Further according to some embodiments, the device further comprises an indicator device in communication with the sensor element, the indicator device being adapted to produce a user-perceptible output corresponding to the sensor output. The indicator device provides an operator/user with real-time status information on the tension between the first and second parts. For example, the sensor element may be adapted to communicate with an indicator device arranged at a proximal end of the endoscope. The indicator device may then provide user-perceptible output to the operator/user, thus allowing the operator/user to monitor and document the correct placement of the device while performing an endoscopic procedure for installing the device. Furthermore, the indicator device, or a further data processing device may also collect the output from the sensor element for the purpose of monitoring, logging, and documenting correct placement and/or proper tensioning of the device. Thereby, reproducibility and/or documentation of the correct placement is further enhanced, and ultimately also patient safety.

Further according to some embodiments of the device, the user-perceptible output is one or more of a visual output, an audible output, a haptic output, and a tactile output. A user-perceptible output gives immediate feed-back to an operator/user of the endoscopic device for installing the gastroenteroanastomosis device. A visual output may be easily provided and may attract immediate attention in a simple and intuitive manner, using e.g. a single light emitter or light emitter array, by varying the parameters of light emitted therefrom in response to a received sensor element output signal. The parameters of the light emission thus varied may include one or more of: a color, an intensity, a blinking frequency, a time sequence pattern, a spatial pattern and the like. A visual output may be also be provided using a display, e.g. presenting a numerical value, or a graphic representation of such a value corresponding to a received sensor element output. A visual output is particularly effective if provided within the field of focus of an operator/user during normal operation of the endoscopic device for installing the gastroenteroanastomosis device. Alternatively or in addition to a visual output, audible, haptic and/or tactile feedback may be provided, thereby further improving the immediate feedback to the operator/user. For example, the operator may not see the visual indication of the value of the sensor element output, but can then hear it or feel it. Advantageously, there may also be a pre-determined set value at which an audible, haptic or tactile output tells the operator that a certain value, e.g. indicative of proper tensioning, has been accomplished.

Thereby user-friendliness of the device and the associated endoscopic instrumentation is improved for an operator/user, and an easy and reliable installation is made possible. This ultimately reduces stress on the patient and also contributes to an in-creased patient safety.

The indicator device and/or the further data processing device may further comprise electronic circuitry and/or programmed instructions allowing for checking the collected sensor element output against pre-determined values for proper tensioning. For example, such predetermined values may be given and/or stored in a memory associated with the indicator device or further processing device as threshold values, upper and/or lower limits defining corresponding ranges for proper tensioning, or the like. Furthermore, the indicator device and/or the further processing device may be configured to compare the sensor element output with the predetermined values for proper tensioning, and provide a user-perceptible warning and/or alarm, if the sensor output approaches and/or exceeds (falls outside) predetermined values for proper tensioning. Thereby user-friendliness of the device and the associated endoscopic instrumentation is further improved for an operator/user, ultimately reducing stress on the patient and contributing to patient safety.

Further according to some embodiments of the device, the indicator device communicates with the sensor through one or more of a wired connection and a wireless connection. Collecting the sensor output by a wired connection or by wireless communication allows to monitor and/or document the proper tensioning of the device during installation in a simple and reliable manner, e.g. by wiring included in an endoscopic catheter for delivering and installing the anastomosis device. A particularly advantageous combination is achieved if the sensor element is arranged (integrated) in the anastomosis device itself and adapted for collecting the sensor element output by wireless communication, e.g. using a wireless transmitter also integrated in the anastomosis device or in the sensor element itself. This allows for monitoring and surveying the correct placement and proper tension even during the period of placement of the device in a patient for the formation of a gastroenteroanastomosis passage, i.e. during the indwelling time of the anastomosis device. Thereby further enhancing patient safety and improving medical documentation of the procedure.

The same and further advantages as discussed herein are also achieved by a further aspect of the invention, which relates to an endoscopic system for use in a minimally invasive procedure for forming a passage across apposed tissue walls from the stomach to a target portion of the small intestines in a patient; wherein the system comprises: a gastroenteroanastomosis device according to any one of the preceding claims; an endoscope adapted for insertion into the stomach at a target site for forming a gastroenteroanastomosis passage, the endoscope comprising an instrument channel extending from a proximal end to a distal end of the endoscope; and an endoscopic catheter adapted for the delivery and placement of the gastroenteroanastomosis device through the instrument channel of the endoscope.

Further according to some embodiments, the system further comprises a sensor element adapted to provide a sensor output indicative of an axial tension forcing (pulling and/or pushing) the first part and the second part towards each other. Furthermore in some embodiments, the sensor element is arranged at a proximal end of the endoscope so as to determine an axial tension between a first control element for the manipulation of the first part and a second control element for the manipulation of the second part.

Further according to some embodiments, the system further comprises an indicator device in communication with the sensor element, the indicator device being adapted to produce a user-perceptible output corresponding to the sensor output. Furthermore in some embodiments, the user-perceptible output is one or more of a visual output, an audible output, a haptic output, and a tactile output. Further according to some embodiments of the system, the indicator device communicates with the sensor through one or more of a wired connection and a wireless connection, such as Bluetooth, BLE, or the like. Further according to some embodiments of the system, the indicator device is arranged at a user end of the endoscope

### BRIEF DESCRIPTION OF THE DRAWINGS

Preferred embodiments of the invention will be described in more detail in connection with the appended drawings, which show in

FIG. 1 schematically, a portion of the gastrointestinal tract of a patient after a first intervention for installing a gastroenteroanastomosis device according to one embodiment of the invention;

FIG. 2 schematically, a portion of the gastrointestinal tract of a patient after a second intervention, after formation of a gastroenteroanastomosis using a device according to one embodiment of the invention;

FIG. 3 schematically, steps of a gastric bypass procedure using a gastroenteroanastomosis device according to embodiments of the invention;

FIG. 4 a side projection view of a gastroenteroanastomosis device according to one embodiment of the invention;

FIG. 5 a perspective view of the first part of the gastroenteroanastomosis device of FIG. 4, deployed through an endoscope;

FIG. 6 a perspective view of the placement of the second part of the gastroenteroanastomosis device of FIG. 4 in the first part of FIG. 5;

FIG. 7 a perspective view of a gastroenteroanastomosis device according to a further embodiment of the invention;

FIG. 8 the gastroenteroanastomosis device of FIG. 7, as seen in a cross-wise cross-section view;

FIG. 9 the gastroenteroanastomosis device of FIG. 7, as seen in a longitudinal cross-section view;

FIG. 10 a side elevation of elements of a second part of a gastroenteroanastomosis device according to a yet further embodiment of the invention, in a collapsed state;

FIG. 11 a side elevation of elements of the second part of the gastroenteroanastomosis device of FIG. 10, in an expanded state;

FIG. 12 a perspective view of the second part of the gastroenteroanastomosis device of FIG. 10, in a collapsed state during introduction;

FIG. 13 a perspective view of the gastroenteroanastomosis device of FIG. 10, in a deployed state, during placement of the second part;

FIG. 14 a perspective view of the gastroenteroanastomosis device of FIG. 10, in a deployed state;

FIG. 15 an example of steps for placing a gastroenteroanastomosis device according to the embodiment of FIG. 4;

FIG. 16 an example of steps for retrieving the gastroenteroanastomosis device of FIG. 15;

FIGS. 17-20 a kit for placement of a gastroenteroanastomosis device according to the embodiment of FIG. 7;

FIG. 21 an example of steps for placing a gastroenteroanastomosis device using the kit of FIGS. 17-20.

FIG. 22 an example of steps for placing a gastroenteroanastomosis device according to the embodiment of FIGS. 23-27;

FIG. 23 a perspective view of a gastroenteroanastomosis device according to a yet further embodiment of the invention in a collapsed state for deployment through an endoscope;

FIG. 24 an exploded perspective view showing components of the gastroenteroanastomosis device of FIG. 23;

FIG. 25 a cross-sectional view of the gastroenteroanastomosis device of FIG. 23 in a deployed state;

FIG. 26 a detail of the gastroenteroanastomosis device of FIG. 25; and in FIG. 27 a further detail of the gastroenteroanastomosis device of FIG. 25.

### DETAILED DESCRIPTION

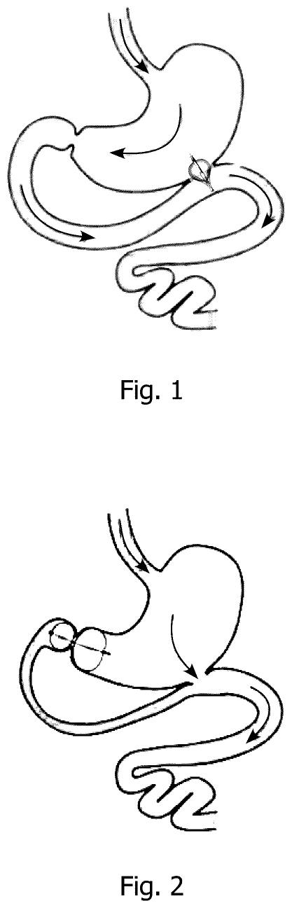

Gastroenteroanastomosis devices according to embodiments of the invention are useful for establishing a fluid connection from the stomach to a portion of the small intestines, thereby at least partially bypassing at least a portion of the duodenum. In particular, devices according to embodiments of the invention allow for performing such a gastrointestinal bypass procedure with as few as two interventions in a patient. FIGS. 1 and 2 schematically illustrate the two interventions required for a procedure for establishing a gastrointestinal bypass using such a gastroenteroanastomosis device according to an embodiment of the invention.

FIG. 1 shows schematically a portion of the gastrointestinal tract of a patient after the first intervention. In the first intervention, a gastroenteroanastomosis device according to one embodiment of the invention is installed to connect the stomach with a target portion of the small intestines. The gastroenteroanastomosis device is placed across two apposed tissue wall portions of the stomach and of the target portion of the small intestines, respectively. The gastroenteroanastomosis device heals the apposed tissue wall portions together along a peripheral region of the device, and forms a passage across the apposed tissue wall portions inwardly of the peripheral region. After the first intervention, the direct passage from the stomach to the target portion of the small intestines is still blocked. The flow of fluids therefore continues to follow the natural path through the pylorus and duodenum as indicated by the arrows, while the gastroenteroanastomosis is being formed.

FIG. 2 shows schematically, a portion of the gastrointestinal tract of a patient after the second intervention. In the second intervention, the formed passage from the stomach to the target portion of the small intestines is opened, and the natural path-way from the stomach to the small intestines through the pylorus is closed. After some time required for the formation of a gastroenteroanastomosis by the device installed in the first intervention, the device is removed from the patient, thereby opening the passage across the two apposed tissue wall portions. The direct passage from the stomach to the target portion of the small intestines at least partially bypasses at least a portion of the duodenum. Finally, a pylorus plug is introduced and positioned in the pylorus. The pylorus plug is for blocking, reducing, or actively controlling flow from the stomach through the pylorus to the duodenum to at least partially redirect the flow of fluids from the stomach to the small intestines through the gastroenteroanastomosis passage, as indicated by the arrows. In a typical procedure, the pylorus plug is configured for blocking the flow through the pylorus, thus redirecting the entire flow through the gastroenteroanastomosis passage. However, in certain patients indications may exist that call for only reducing, or even actively controlling the flow from the stomach to the small intestines through the pylorus, thus only partially redirecting the flow of fluids from the stomach to the small intestines through the gastroenteroanastomosis passage. A volume of the proximal portion of the pylorus plug, which is located within the stomach, may furthermore be selected and/or adjusted, in order to reduce the remaining space available in the stomach after conclusion of the procedure. The gastroenteroanastomosis passage bypasses the portion of the small intestines located between the pylorus and the target region of the small intestines. Depending on where the target portion is located in the small intestines, the gastroenteroanastomosis passage bypasses only a portion of the duodenum, namely an upstream portion of the duodenum between the pylorus and the target region, or the entire duodenum. Using a minimally invasive procedure of the type as described here with respect to FIGS. 1 and 2, the gastric bypass may be reversed in a simple manner, by removing the pylorus plug again to allow for a flow of fluids via the pylorus and the duodenum. Depending on the specific indications for a given patient, the reversing procedure may either leave the gastroenteroanastomosis passage open, or close the passage again, e.g. in an endoscopic surgical procedure.

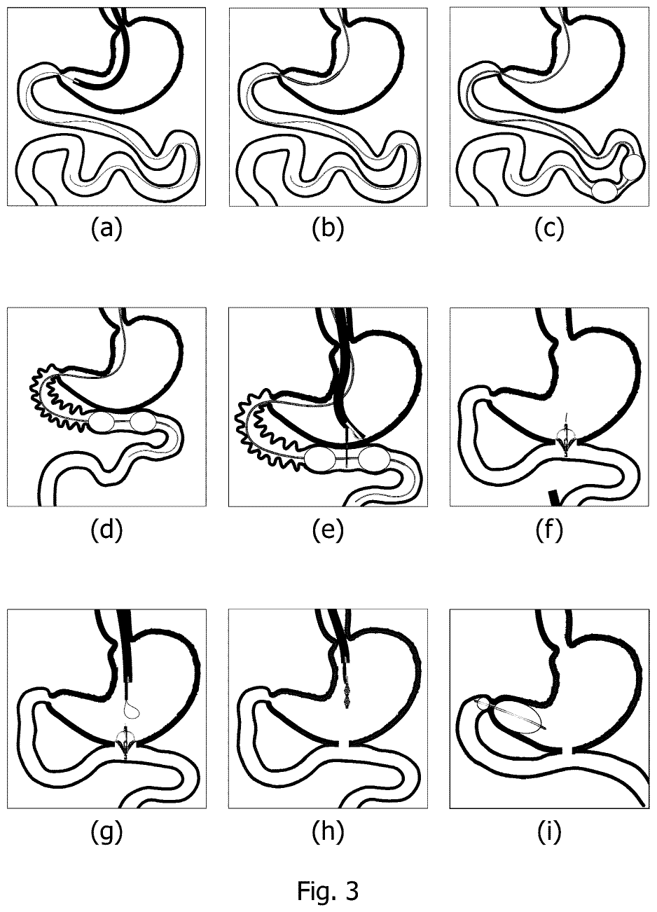

FIG. 3 gives a more detailed example of the use of a gastroenteroanastomosis device according to some embodiments in such a minimally invasive gastrointestinal bypass procedure with two interventions, where FIG. 3(a)-(f) shows steps in the first intervention, and FIG. 3(g)-(i) shows steps in the second intervention of the procedure. The entire procedure is performed transorally. First, a guidewire is introduced endoscopically through the mouth, esophagus, stomach, and through the pylorus into the small intestines, reaching beyond a target portion of the small intestines (step (a)). Then a double-balloon catheter is introduced following the guidewire, placing the two balloons around the target portion and filling them with fluid. The two balloons engage and seal off a volume including the target portion of the small intestines (steps (b) and (c)). The double balloon catheter is then pulled back to place the target portion in apposition with the stomach, and the target portion embraced by the two balloons is also filled with a fluid, such as saline solution, which is easily detect-able by e.g. an ultrasonic endoscope (step (d)). The gastroenteroanastomosis device is then placed and installed to connect a tissue wall portion of the stomach with a tissue wall portion of the target portion of the small intestines (steps (e) and (f)). This concludes the first intervention, leaving the device in the patient to form the gastroenteroanastomosis between the stomach and the small intestines (step (f)). After sufficient amount of time allowing for the anastomosis to form, the device is captured, e.g. using a snare, and collapsed in order to be retrieved from the patient endoscopically, e.g. through the instrument channel of an endoscope, thereby opening the gastroenteroanastomosis passage formed (steps (g) and (h)). The second intervention of the procedure is concluded by introducing and installing a pylorus plug, thereby at least partially bypassing at least a portion of the duodenum as discussed above (step (i)).

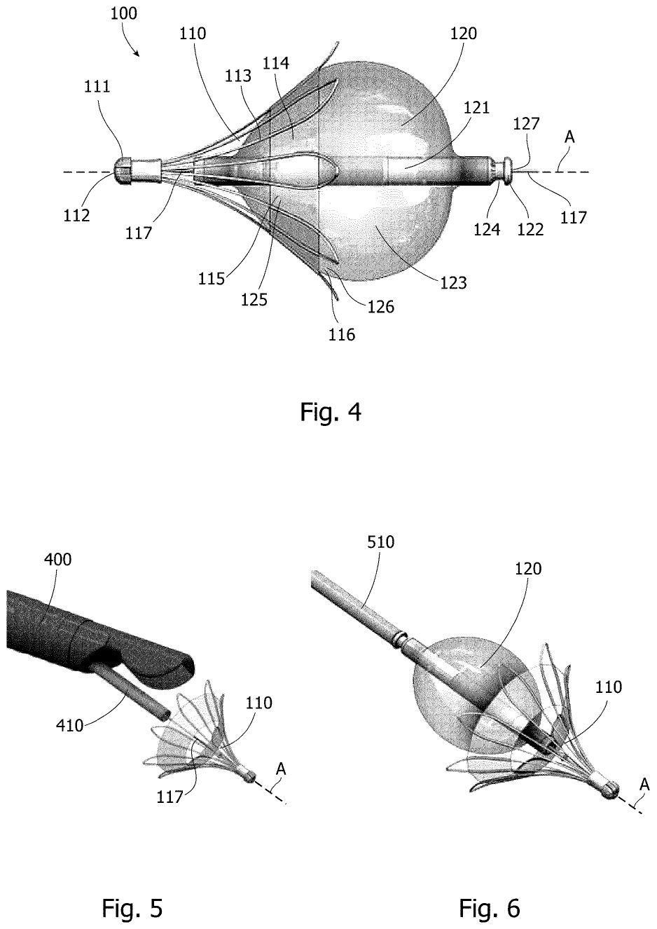

Turning now to FIGS. 4-14, different embodiments of gastroenteroanastomosis devices according to the invention are now described. Generally, a gastroenteroanastomosis device according to any of the embodiments disclosed herein has a collapsed state adapted for the minimally invasive introduction of the device into a patient, and a deployed state adapted for anastomotic engagement of apposed tissue walls when the device is placed at the anastomosis site.

FIGS. 4-6 show a gastroenteroanastomosis device 100 according to one embodiment. In FIG. 4, the gastroenteroanastomosis device 100 is seen in its deployed state. The device 100 has a first part 110 connected to a second part 120 via a centrally arranged mechanical connection adapted for maintaining a fixed placement of the first and second parts 110, 120 with respect to each other. The mechanical connection may be configured to comprise one or more pre-determined settings for the fixed placement, such as in a snap-fit engagement with one or more snap positions. The device 100 is introduced into a patient and placed at the anastomosis site using a minimally invasive intervention, where the two parts 110, 120 are introduced and placed in sequential order as indicated by FIG. 5 and FIG. 6. FIG. 5 shows the first part 110 in an expanded state after introduction through the instrument channel of an endoscope 400 by means of a catheter 410. The first part is connected to a guidewire 117 extending all the way to an opposite end thereof outside the patient. FIG. 6 shows introduction of the second part 120, pushed by a catheter 510 to approach the first part 110 along the guidewire 117.

The first part 110 is for placement on the enteric side, and is adapted to engage the tissue wall of the small intestines at the target region from the inside of the small intestines. The second part 120 is for placement on the gastric side, and is adapted to engage the stomach wall from the inside of the stomach. The first and second parts 110, 120 are connected to each other by a connection penetrating the engaged tissue wall portions in a central portion thereof, through an opening with a small diameter, and enclosed by circumferential engagement surfaces 115, 125 as further detailed in the following.

In the deployed state, the second part 120 is seated against the first part 110, the two parts engaging the two apposed tissue walls between them. When installing the device 100, the mutual position of the first and second parts 110, 120 is adjusted according to a desired pressure distribution applied to the engaged tissue walls. Once the two parts 110, 120 are installed, their placement with respect to each other is typically fixed until the device eventually is removed in a second intervention, after formation of the gastroenteroanastomosis passage, as described above.

An axial direction of the device 100 is defined in its deployed state by a central axis A extending from a distal end 111 to a proximal end 121. When the device is in the collapsed state, the central axis A and the axial direction are also parallel to the direction of advancement for endoscopic insertion into a patient. When the device 100 is placed in the patient for forming a gastroenteroanastomosis passage, the axial direction corresponds to the direction of penetration of the tissue walls. When installed, the axial direction of the device 100 is essentially perpendicular to the general direction of the tissue walls at the site of placement. Transverse directions of the device 100 are defined as parallel to a plane that is perpendicular to the axial direction, i.e. perpendicular to the central axis A. Radial directions are transverse directions intersecting the central axis A.

At the distal end 111, the first part 110 of device 100 has a tip with a cutting element 112 facilitating penetration of the tissue walls during installation. The cutting element 112 may be of any suitable type as known in the art, such as a needle, knife edge, or preferably an electrically heated cutting wire. The first part 110 of device 100 furthermore has radially extending elements 113, here provided as wire loops originating at the distal end 111 and shaped to flare out from the central axis A like petals of a flower, thereby forming a gradually (such as conically or similarly diverging) opening inner surface for receiving the second part 120 therein. The radially extending elements 113 are collapsible to fit within a radial circumference of a given channel allowing for the minimally invasive delivery of the first part, e.g. through an instrument channel of an endoscope. The wire loops may be made of any suitable material allowing for assuming a collapsed state and an extended state. For example, the wire loops may be produced as shapes that are mechanically biased towards an extended state, which may be held together in the collapsed state by a sleeve, and which may be released to their extended state by removing the sleeve upon introduction and placement. The sleeve may be of any suitable construction, such as a foil wrapping that can be broken and removed, or a wall of a catheter used for delivery of the first part 110. The wire loops may be made of any suitable biocompatible material. The wire loops may also comprise memory materials, such as nitinol, configured to assume a collapsed state, and upon activation an extended state, where activation is after introduction and placement of the first part in its enteric location.

A guidewire 117 extends from the distal end 111 of the device 100 along the central axis A of the device 100 to an opposite end of the guidewire outside the patient. The distal portion of the guidewire 117 is part of the connection connecting the first and second parts 110, 120 of the device 100 to each other. The second part 120 has a centrally arranged stem 121 with an inner channel 127 for receiving the guidewire 117 of the first part 110 therein, thereby allowing for (minimally invasive) delivery of the second part 120 along the guidewire 117 to meet the first part 110. The device further comprises a locking means (not shown) adapted for keeping the two parts 110, 120 in fixed placement with respect to each other. Once the second part 120 is in place it may be locked in fixed engagement with respect to the first element using the locking element (not shown). The stem 121 thus cooperates with the distal portion of the guidewire 117 to link the first and second parts 110, 120 together. The stem 121 is thus also part of the connection connecting the first and second parts 110, 120.

The central stem 121 carries an inflatable body 123, which when inflated presents a generally convex, radially outward facing surface towards a generally concave, radially inward facing surface of the first part 110. According to some embodiments, the inflatable body may comprise a balloon. According to some embodiments, the inflatable body 123 may comprise a compressible and/or self-inflating material, such as a foam material, which may be compressed for introduction through the esophagus, and configured to expand in the stomach, upon delivery close to the site of placement. According to some embodiments, the inflatable body may also be replaced with a resilient body, which is shaped and dimensioned for transoral delivery to the stomach through the esophagus.

The concave inner surface of the first part 110 comprises a first engagement region 115 for seating a cooperating second engagement region 125 on the convex outer surface of the second part. The concave inner surface of the first part 110 further comprises a first peripheral region 116 located peripherally outward of the first engagement region 115, and facing a cooperating second peripheral region 126 on the convex outer surface of the second part 120. The convex outer surface of the inflatable body 123 of the second part cooperates with the concave inner surface of the first part to guide the two parts into proper alignment with respect to each other, such that the first engagement surface 115 is aligned vis-á-vis the second engagement surface 125, and the first peripheral surface 116 is aligned vis-á-vis the second peripheral surface 126.

The cooperation of a concave surface on one of the first and second parts 110, 120 with a convex surface on the other one of the first and second parts 110, 120 facilitates self-alignment of the first and second part with respect to each other. This ensures proper alignment of the engagement surfaces 115, 125 and of the peripheral surfaces 116, 126 with respect to each other-both when seating the second part 120 against the first part 110 during placement of the device 100, and for properly keeping the parts 110, 120 of the device 100 in place throughout the period of forming the gastroenteroanastomosis. Furthermore, the number of degrees of freedom to be controlled by the practitioner for installing the device is reduced. As a result, an efficient installation, and a safe device for reliably forming a gastroenteroanastomosis passage is achieved.

The inflatable body 123 is delivered in a deflated, collapsed state, and inflated upon placement of the second part 120 on the gastric side to engage the apposed tissue walls against the extended first part 110 already placed on the enteric side. The inflatable body 123 is then inflated to an operating pressure and sealed off to stay pressurized during formation of the gastroenteroanastomosis passage. After formation of the gastroenteroanastomosis passage, the inflatable body 123 is deflated again, e.g. by simply puncturing the inflatable body 123, and thus collapses for retrieval of the device 100 in a minimally invasive intervention.

The proximal end 122 of the stem 121 has a coupling element 124 adapted for engagement by an endoscopic instrument to facilitate reliable retrieval of the device 100 after formation of the gastroenteroanastomosis passage. For example, the proximal end 122 of the stem 121 may have a coupling groove 124 for capture by a snare.

In the embodiment shown in FIGS. 4-6, the wire petals are at least partially wrapped with a membrane 114. The membrane 114 is stretched out in the deployed state of the device 100, thereby confining the opening angle of the flower cone. The membrane also serves to confine the inflation of the inflatable body, and to distribute the forces between the two parts in a circumferentially closed ring. The inner surface of the first part 110 has a circumferential first engagement region 115 adapted for seating a cooperating circumferential second engagement region 125 of the second part 120. When the device 100 is installed at the anastomosis site, the first and second engagement regions 115 and 125 engage the tissue walls with ischemic pressure at least along a circumferentially closed contour, thereby causing necrosis of the tissue, eventually opening a passage defined by the circumferential contour of the cooperating engagement surfaces 115, 125. In other words, upon installation, the cooperating first and second engagement regions of the first and second part are placed with respect to each other so as to define an ischemic pressure region with a circumferential contour of the desired passage opening. Furthermore, the cooperating first and second parts 110, 120 are flexible and/or pressurized so as to maintain an ischemic pressure throughout the period of installation of the device, and to accommodate any decrease in tissue thickness during the necrosis process.

Peripherally adjacent thereto in a radially outward direction, and enclosing the engagement region, a first peripheral region 116 of the first part 110 cooperates with a second peripheral region 126 of the second part 120 to form a region of gradually diverging surfaces. In this peripheral region, the distance between the cooperating surfaces 116, 126 of the first and second parts 110, 120 gradually increases in a radially outward direction, from a distance between the first and second engagement regions 115, 125, which is adapted for ischemic engagement, to a distance equal to, or exceeding, the sum of thicknesses of the uncompressed tissue walls. When the device 100 is installed at the anastomosis site, the gradually diverging surfaces of the first and second peripheral regions 116, 126 gradually release the pressure applied to the tissue walls from an ischemic pressure between the cooperating engagement surfaces 115, 125, to no pressure when the distance between the first and second peripheral surfaces 116, 126 exceeds the combined thickness of the uncompressed tissue walls. The cooperating first and second peripheral regions 116, 126 of the first and second part are thus adapted to keep the tissue walls in sealed contact with each other in a region peripherally enclosing the ischemic pressure region. The peripheral seal is provided right from the beginning, upon introduction and placement in the patient, and to provide a region of non-ischemic pressure, so that the tissue walls can heal together in this region, while a passage forms by necrosis in the engagement region enclosed by the peripheral seal. Thereby, a particularly safe way of forming the gastroenteroanastomosis passage is provided, where leaks of gastric fluids into the abdominal cavity are suppressed right from the beginning.

As mentioned above, the gradually diverging surfaces 116, 126 in the peripheral region provide a gradual pressure zone ensuring that the pressure applied to the tissue walls gradually decreases from an ischemic pressure in the engagement region between the first and second engagement surfaces 115, 125, to safely reach a non-ischemic regime where the tissue walls can heal together. The gradual pressure zone thus ensures a particularly safe and reliable formation of the peripheral seal for the gastroenteroanastomosis. Furthermore, the degree of divergence of the cooperating first and second peripheral surface regions 116, 126 may be determined, thus determining the width of the gradual pressure zone formed between these peripheral surface regions 116, 126, as seen in a transverse direction along the surface regions 116, 126. By determining the width of the gradual pressure zone where a non-ischemic pressure is applied, the width of the safe sealing seam surrounding the opening of the gastroenteroanastomosis passage is determined. Typically, the width of the gradual pressure zone is between 2 mm and 2 cm; advantageously, the width of the gradual pressure zone is between 3 mm and 15 mm; and further advantageously, the width of the gradual pressure zone is between 4 mm and 1 cm. The gradual pressure zone thus provides a reliable method for configuring the device to provide a safe and reliably sealed gastroenteroanastomosis.

The engagement region formed by the cooperating first and second engagement surfaces 115, 125 also encloses the centrally arranged mechanical connection connecting the first and second parts 110, 120, thereby sealing the penetration during the necrotic formation of the passage. Furthermore, since the peripheral regions 116, 126 enclose the engagement regions 115, 125, they also enclose the centrally arranged penetration for the connection. The small initial penetration produced by the centrally arranged mechanical connection is thus also sealed off by the non-ischemic peripheral seal provided by the cooperating first and second peripheral regions 116, 126. The risk of leakage of gastric fluids into the abdominal cavity is therefore minimized.

The embodiment shown in FIG. 4-6 has rotational symmetry around the central axis A of the device 100, thereby being adapted to form an essentially circular gastroenteroanastomosis passage. A circular symmetry has the advantage that the pressure distribution is essentially invariant in a circumferential direction along the contours defining the engagement and peripheral regions 115, 125, 116, 126. Thereby a well-controlled and predictable performance is achieved for the device 100 having a first part 110 with a foldable wire frame 113 and a cooperating second part with an inflatable body 123. However, other cooperating shapes of the foldable wire frame 113 and the inflatable body 123 are generally conceivable, such as a shape that is elongated in one transverse direction (e.g. longitudinal) as compared to another transverse direction (e.g. cross-wise), thus providing correspondingly elongated engagement and peripheral surfaces and resulting in an elongated shape of the gastroenteroanastomosis passage formed by such an elongated device. Furthermore, the elements composing the wire frame may be shaped and dimensioned to minimize transverse dimensions of the wire frame in the collapsed state. For example, the flower petals of the above-mentioned flower-like embodiment may be of different length in order to reduce a transverse dimension in its collapsed state, e.g. when folded inside a delivery sleeve.

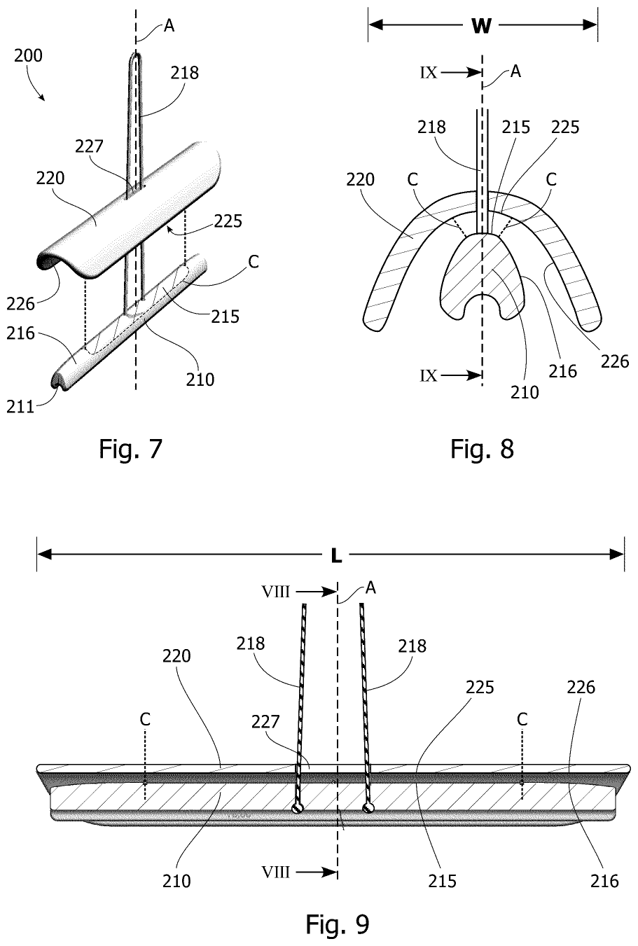

Turning to FIGS. 7-9, a gastroenteroanastomosis device 200 according to a further embodiment is now discussed. The gastroenteroanastomosis device 200 is bar-shaped and elongated in one direction. The device 200 is thus adapted for the formation of an elongated gastroenteroanastomosis passage akin to a buttonhole rather than a circular passage. The device 200 has a first part 210 and a cooperating second part 220 connected by flexible connection elements 218. The flexible connection elements 218 are adapted for pulling the first and second parts 210, 220 towards each other. In the deployed state, the second part 220 is seated against the first part 210, the two parts engaging the two apposed tissue walls between them.

When installing the device 200, the mutual position of the first and second parts 210, 220 is adjusted according to a desired pressure distribution applied to the engaged tissue walls. Once the two parts 210, 220 are installed, their placement with respect to each other is typically fixed until the device is removed in a second intervention, after formation of the gastroenteroanastomosis passage, as described above. A locking element (not shown) is adapted to lock the first and second parts 210, 220 in a fixed placement with respect to each other.

An axial direction is defined by a central axis A along the direction of the connection between the first and second parts 210, 220 of the bar-shaped device 200. In the deployed state of the device 200, the central axis A is perpendicular to the direction of elongation of the bar-shape. Transverse directions of the device are defined as perpendicular to the axial direction, wherein a longitudinal direction of the device is along a generally lengthwise direction of the intestinal tube according to the intended placement of the device at the target region (and perpendicular to the axial direction), and a crosswise direction is from side to side of the intestinal tube, perpendicular to the longitudinal direction (and perpendicular to the axial direction). FIGS. 8 and 9 provide cross-sectional views of the device in a deployed state. FIG. 8 shows a transverse cross-section of the device 200 as seen in a cross-wise cut plane along line VIII-VIII in FIG. 9. FIG. 9 shows a longitudinal cross-section of the device 200 as seen in a longitudinal cut plane along line IX-IX in FIG. 8.

The first part 210 is centrally attached to the flexible connection elements 218, and is tiltable around a cross-wise axis between a collapsed state, where the direction of elongation of the first part 210 is parallel to the axial direction, and an extended state, where the direction of elongation of the first part 210 is perpendicular to the axial direction (and perpendicular to the longitudinal direction). The first part 210 is for enteric placement and is inserted from the gastric side through a small opening penetrating the apposed tissue walls in the collapsed state and then tilted like a toggle catch into the extended state to draw the enteric wall towards the gastric wall.

The connection elements 218 pass through a central opening 227 in the cooperating second part 220. The cooperating second part 220 is for gastric placement and is introduced along a guidewire (not shown), which is connected to a proximal end of the connection 218 and passed through the central opening 227 of the second part 220. Like the first part 210, the second part 220 may also be tilted about a cross-wise axis for delivery (direction of elongation parallel to the axial direction) and placement (direction of elongation perpendicular to the axial direction). The second part 220 is thus placed to press the gastric wall towards the enteric wall, against the first part 210. Advantageously, the connection elements 218 may be arranged in a triangular fashion, e.g. wires that are kept in tension during the mounting procedure, and may thus act as a steering feature in the elongated central opening 227 in the cooperating second part 220. The connection elements 218 may thus be adapted to provide a steering interaction with the central opening 227 to align the two parts when pulled/pushed together.

The first and second parts 210 and 220 engage the apposed tissue walls with cooperating surfaces facing towards each other, each comprising an engagement region 215, 225 enclosed by a peripheral region 216, 226. Upon placement, the cooperating engagement regions 215, 225 of the first and second parts 210, 220 are pulled together and fixed at a distance so as to apply an ischemic pressure to the apposed tissue walls causing necrosis for forming an opening. When placing the parts, they may e.g. be pulled together to a predetermined position or to a predetermined force. Placement may be aided by a mechanical connection comprising one or more pre-determined settings for the fixed placement, such as in a snap-fit engagement with one or more snap positions, akin to cable ties, thereby allowing for easy adaptation to tissue thickness variations.

At the same time, the cooperating peripheral regions 216, 226 provide diverging surface gradually increasing the distance to a value equal to or exceeding the sum of the thicknesses of the uncompressed tissue walls, thereby gradually decreasing the applied pressure from an ischemic regime at the engagement regions, through a non-ischemic regime to no pressure as seen in a peripherally outward direction. The cooperating peripheral surfaces 216, 226 thus provide a well-defined region of contact between the apposed tissue walls in a non-ischemic regime, which fully encloses the engagement regions 215, 225 where the opening is formed. The cooperating peripheral surfaces 216, 226 thus ensure right from the beginning of the procedure a reliable seal around the is chemically engaged tissue portions, where the opening is slowly formed by necrosis, and at the same time creates a seam of the tissue walls healed together around the opening.

The first part 210 has a generally convex shaped cooperating surface facing towards a generally concave shaped cooperating surface of the second part 220 adapted to receive the first part 210 therein. The engagement regions 215, 225 may be outlined by a contour C enclosing respective central portions of the cooperating surfaces of the first and second parts 210, 220 of the device 200. The peripheral regions 216 and 226 are located outwardly adjacent to the contour C of the engagement regions 215, 225. As best seen on FIG. 8, in a transverse direction, the engagement regions 215, 225 located inside the contour C are similarly shaped and sized to follow each other at a distance configured for applying a pressure leading to ischemic engagement of the apposed tissue walls clamped between the engagement regions 215, 225. Adjacent to the contour C, in an outward direction away from the engagement regions 215, 225, the peripheral regions 216, 226 are shaped to diverge from each other, thus increasing the distance between the peripheral regions from a distance corresponding to ischemic engagement at the contour C to distances corresponding to a non-ischemic regime, and eventually a distance exceeding the sum of the typical thicknesses of the uncompressed tissue walls at the outer edge. Correspondingly, as best seen in FIG. 9, in a longitudinal direction, the engagement regions 215, 225 located inside the contour C are essentially parallel, following each other at a distance configured for applying a pressure leading to ischemic engagement of the apposed tissue walls clamped between the engagement regions 215, 225. Outside the contour C, the peripheral regions 216, 226 are shaped to diverge from each other to increase the distance between them for gradually releasing the pressure applied to the apposed tissue walls. In the embodiment shown in FIGS. 7-9, this is achieved by simply extending the engagement region 225 to also maintain the straight shape throughout the peripheral region, whereas at the same time the first part tapers off in a longitudinal direction from the contour C around the engagement region 215 towards the outer ends, thereby gradually increasing the distance between the peripheral regions 216, 226 in an outward longitudinal direction, from the contour C towards the outer ends of the device 200. The peripheral region thus provides a gradual transition from an ischemic regime at the contour of the engagement regions 215, 225, to no pressure at the outer edges and ends of the device 200, thereby providing a well-defined non-ischemic regime for forming a reliable seal around the passage formed by necrosis between the engagement regions 215, 225.

The gastroenteroanastomosis device 200 has an overall length L as seen in the longitudinal direction and an overall width W as seen in the cross-wise direction. The elongation of the device 200 may be described by its aspect ratio R=L/W. Correspondingly, the elongated engagement regions 215, 225 configured for forming the corresponding passage opening by necrosis may be described by the length I and the width w of the cooperating regions 215, 225, with an aspect ratio r=I/w.

The size or gauge of the opening formed by necrosis within the bounds of the peripheral contour C of the engagement regions 215, 225, may be described independent of its actual shape by the length of its perimeter s. The opening is permanently sealed along its perimeter by a peripheral seam, where the apposed tissue walls of the stomach and the target region of the small intestines are healed together by anastomosis. A width of the seal as seen in a radial direction perpendicular to the edge of the opening is determined by the width of the peripheral regions 216, 226 providing a non-ischemic engagement of the apposed tissue walls during formation of the gastroenteroanastomosis passage.

The two cooperating parts 210, 220 of the embodiment 200 shown in FIGS. 7-9 are made of a material that is relatively stiff as compared to the flexibility of the tissue walls. In particular, the cooperating parts are configured to be stiff enough to transfer the centrally applied pressure from around the connection 218 towards the ends and edges of the device, so as to maintain an ischemic pressure at the designated contour of the cooperating engagement regions 215, 225.

A foremost advantage of embodiments akin to that shown in FIGS. 7-9 is the simplicity of construction, facilitating a relatively simple production at low cost, and easy handling for introduction and placement of the device. At the same time, a reliable seal is achieved in a simple manner, due to the presence of the gradually diverging peripheral surface regions, which peripherally enclose a central engagement region where the passage opening is formed by necrosis. Furthermore, the elongated shape of the device that is aligned with the longitudinal direction along the general direction of the small intestines in the target region benefits forming a passage with a relatively large perimeter s, and thus a relatively large opening as compared to the width of the tube of the small intestines. Furthermore, during placement, the first part 210 is received in the second part 220 in a self-aligning manner. This is achieved by providing a second part 220 that is trough shaped to present concavely shaped engagement and peripheral regions 216, 226 towards convexly shaped engagement and peripheral regions 215, 225 of the first part 210. The self-aligning property of the convex surface regions with the cooperating concave surface regions is also beneficial for maintaining the correct placement of the first and second parts with respect to each other during the entire period of forming the gastroenteroanastomosis passage. The correct placement ensures that the device applies the correct pressure distribution to the apposed tissue walls clamped between the first and second parts 210, 220. Thereby, reliability and safety of the intervention is further enhanced.

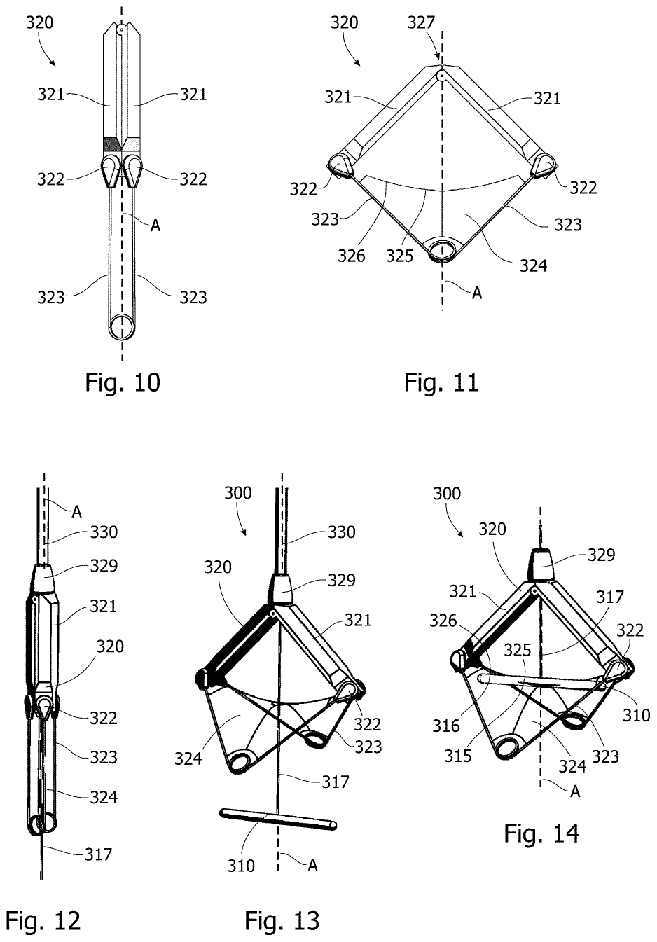

Turning to FIGS. 10-14, a gastroenteroanastomosis device 300 according to a yet further embodiment is now discussed. The gastroenteroanastomosis device 300 is elongated in one direction, like the embodiment 200 discussed above with reference to FIGS. 5-9. The device 300 is thus also adapted for the formation of an elongated gastroenteroanastomosis passage akin to a buttonhole rather than a circular passage. The device 300 has a first part 310 and a cooperating second part 320 connected by a flexible guidewire 317. A distal portion of the guidewire 317 is adapted for pulling the first and second parts 310, 320 towards each other. In the deployed state, the second part 320 is seated against the first part 310, the two parts engaging the two apposed tissue walls between them. When installing the device 300, the mutual position of the first and second parts 310, 320 is adjusted according to a desired pressure distribution applied to the engaged tissue walls. Once the two parts 310, 320 are installed, their placement with respect to each other is typically fixed until the device is removed in a second intervention, after formation of the gastroenteroanastomosis passage, as described above. A locking element 329 is adapted to lock the first and second parts 310, 320 in a fixed placement with respect to each other.

An axial direction is defined by a central axis A along the direction of the connection between the first and second parts 310, 320 of the elongated device 300. The direction of elongation is defined as the direction of elongation of the cooperating surfaces adapted to clamp the apposed tissue walls between them. The device 300 shown in FIGS. 10-14 has a first part 310 shaped as a simple elongated bar with a convexly rounded outer surface, which at its center is attached to the guidewire 317 extending in the axial direction. The direction of elongation of the first part 310 corresponds to the direction of elongation of the device 300. In the deployed state of the device 300, the central axis A is perpendicular to the direction of elongation of the device 300. Transverse directions of the device 300 are defined as perpendicular to the axial direction, wherein a longitudinal direction of the device 300 is along a generally lengthwise direction of the intestinal tube according to the intended placement of the device 300 at the target region (and perpendicular to the axial direction), and a crosswise direction is from side to side of the intestinal tube, perpendicular to the longitudinal direction (and perpendicular to the axial direction).

FIGS. 10 and 11 give a side elevational view of the second part 320 of the device 300 in a collapsed and deployed state, respectively. FIGS. 12-14 give perspective views of the device 300, wherein: FIG. 12 shows the second part 320 in a collapsed state for delivery along the guidewire 317 leading to the first part 310 (not seen in FIG. 12); FIG. 13 shows the second part 320 in an expanded state approaching the first part 310, also seen in an expanded state, following the guidewire 317; and FIG. 14 shows the device 300 in the deployed state, where the second part 320 is seated against the first part 310, and locked in place by locking element 329 being fixed to the guidewire 317.

The first part 310 is centrally attached to the guidewire 317, and is tiltable around a cross-wise axis between a collapsed state, where the direction of elongation of the first part 310 is parallel to the axial direction, and an extended state, where the direction of elongation of the first part 310 is perpendicular to the axial direction (and perpendicular to the longitudinal direction). The first part 310 is for enteric placement and is inserted from the gastric side through a small opening penetrating the apposed tissue walls in the collapsed state and then tilted like a toggle catch into the extended state to draw the enteric wall towards the gastric wall.

The guidewire 317 passes through a central opening 327 in the cooperating second part 320. The cooperating second part 320 is for gastric placement and is introduced along the guidewire 317. After introduction, the second part 320 is expanded and placed to press the gastric wall towards the enteric wall, against the first part 310.

The second part 320 has two articulated legs 321, with the articulated joint arranged at the proximal end of the legs. In the collapsed state, the two legs 321 are folded onto each other and parallel to the axial direction, as seen in FIG. 10. A membrane 324 is arranged between the legs 321. The membrane 324 is configured to unfold in transverse directions when the legs 321 of the second part 320 spread apart. The membrane 324 provides a surface with second engagement and peripheral regions 325, 326 for receiving cooperating first engagement and peripheral regions 325, 326 on the first part 310. The legs 321 may be moved from the collapsed state into the expanded state by any suitable actuation mechanism. Advantageously, the legs are mechanically biased into the expanded state, e.g. by means of springs to facilitate reliable unfolding of the membrane 324 upon introduction of the second part 320, prior to placement against the first part 310. During minimally invasive introduction, the legs 321 may be retained in the collapsed state by any suitable means, which may be released after introduction, such as a catheter wall, a sleeve that can be removed by sliding it away, a foil wrapping that may be ripped or dissolved, or the like.

In the embodiment seen in FIGS. 10-14, the distal ends of the legs 321 are connected to each other via springs 323 carrying the membrane 324. The springs 323 are configured to bias the legs 321 towards a spread position for expanding the second part 320 and unfold the membrane 324. Each spring 323 has two arms which are connected to each other by a helical spring at a distal end thereof. The proximal ends of the two arms are hinged to a respective one of the two arms by pivot joints 322 arranged at a distal end of the legs 321. The arms of the springs 323 extend in a distal direction away from the legs 321 joined at a distal end by a spring element, such as a helical spring element. In the collapsed state of the second part 320, the arms of the springs 323 are also arranged essentially parallel to the axial direction. The pivot joints 322 are in a transverse direction inclined with respect to the cross-wise oriented axis of the articulated joint connecting the two legs 321, and pointing away from each other in a radial direction. As a consequence of this configuration, the distal ends of the spring arms are forced outwardly in a cross-wise direction, when the legs are spread in a longitudinal direction. The membrane 324 is thus unfolded to a length and a width in the expanded state that largely exceed the diameter of the device 300 in the collapsed state as seen in a transverse cut-plane perpendicular to the axial direction. FIGS. 11, 13, and 14 show the second part 320 in its expanded state, where the membrane 324 to exhibit a saddle shaped cooperating surface facing towards the first part 310, with a concave profile in a cross-wise direction, and a convex shape in the longitudinal direction. As mentioned above, FIG. 12 shows introduction of the second part 320 along the guidewire 317 in the collapsed state, by means of a hollow pushing rod 330 acting via locking element 329 on the proximal end of the second part 320. FIG. 13 shows how the second part 320, pushed by rod 330, approaches the first part 310 in the expanded state, with the membrane 324 unfolded. FIG. 14 shows the device 300 in the deployed state, where the cooperating surfaces of the unfolded membrane 324 and of the first part 310 are forced towards each other and locked in place when the locking element 329 is fixed to the guidewire 317. By forcing the first and seconds parts 310, 320 towards each other, a central region of the flexible membrane 324 essentially conforms to the shape of the first part (via the intermediate of the engaged tissue walls). The interaction of the surfaces thus provides centrally arranged engagement regions 315, 325 cooperating to provide an ischemic pressure to the apposed tissue walls engaged by these regions, and further provides peripheral regions 316, 326 enclosing the engagement regions 315, 325 in peripherally outward directions. The peripheral regions 316, 326 diverge to gradually increase the distance between them. The diverging peripheral surface regions 316, 326 gradually reduce the pressure applied to the apposed tissue walls from an ischemic regime to no pressure, passing through a non-ischemic regime. The non-ischemic regime provides a peripheral seal where the tissue walls are pressed together in a non-ischemic regime to allow the tissue walls to heal together and form a permanently sealed seam enclosing the passage opening formed by necrosis in the engagement regions 315, 325.

The above-mentioned advantages related to the elongated shape also apply to the elongated device with a foldable second part as discussed herein, with reference to FIGS. 10-14. Furthermore, the above-mentioned advantages of the cooperation between a convex and concave surface regions shaped for providing a self-aligning property facilitating correct placement, and for subsequently ensuring that the device stays correctly in place, also apply to the elongated device with a foldable second part as discussed herein, with reference to FIGS. 10-14. A further advantage of the device 300 seen in FIGS. 10-14 is that the cross-wise extension of the second part further improves self-alignment during placement, and improves keeping the device in correct placement during formation of the gastroenteroanastomosis passage.

As seen in the embodiments discussed above with reference to FIGS. 4-14, the first part for enteric placement presents a low cross-sectional area as seen in a cross-wise cut-plane perpendicular to the longitudinal direction, as compared to a cross-sectional area of the small intestinal tube at the target region. Thereby, the risk of obstruction of the target region of the small intestines by material transported from the pylorus via the duodenum through the target region of the small intestines is largely reduced. This is beneficial for patient safety, since passage of material still occurs through pylorus and duodenum along the intestinal tube during presence of the gastroenteroanastomosis device at the target site while forming the passage. This is so when using a device according to embodiments of the invention in a gastric bypass procedure, because the gastroenteroanastomosis passage is only opened after the tissue walls portions forming the peripheral seal are permanently healed together to avoid any leakage of gastric fluids into the abdominal cavity, which also benefits patient safety.

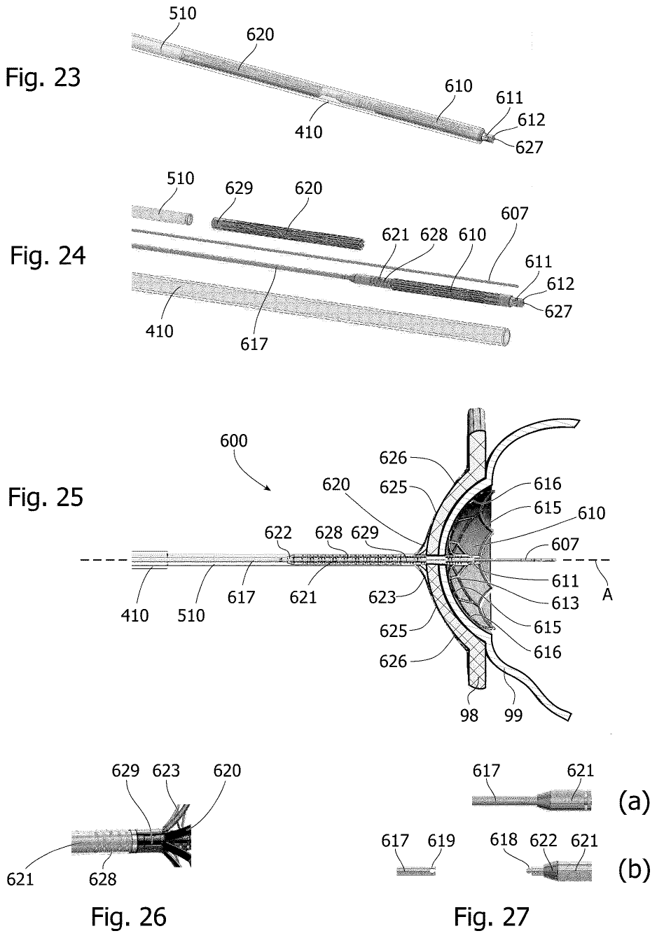

Referring to FIGS. 23-27, a gastroenteroanastomosis device 600 according to a yet further embodiment is now described. FIG. 23 shows the gastroenteroanastomosis device 600 in a collapsed state arranged in an endoscopic catheter 410 for endoscopic deployment. A distal portion of the catheter 410 is shown in FIG. 23. The gastroenteroanastomosis device 600 comprises distally a first part 610 and proximally a second part 620, which are here seen in the collapsed state with transverse dimensions so as to fit in a lumen of the endoscopic catheter 410. The endoscopic catheter 410 is adapted for endoscopic delivery, e.g. through an instrumentation channel of an endoscope, such as the endoscope 400 seen in FIG. 5. A distal tip 611 of the gastroenteroanastomosis device 600 comprises a cutting element facilitating penetration of the tissue walls during installation, such as an electrically powered cutting element 612. The cutting element 612 may be of any suitable type as known in the art, such as a needle, knife edge, or preferably an electrically heated cutting wire. The tip 611 with the cutting element 612 projects from a distal end of the endoscopic catheter 410. Furthermore, a pushing tube 510 is arranged inside the endoscopic catheter 410 proximally of the second part 620. The pushing tube 510 extends all the way to a proximal end of the endoscopic catheter 410 for manipulation by a user.

The various components of the arrangement of FIG. 23 are further discernible from the exploded perspective view in FIG. 24 showing a distal portion of the endoscopic catheter 410, a distal portion of the pushing tube 510, the collapsed second part 620, and a distal portion of a stem delivery tube 617 carrying a detachable stem 621 with the collapsed first part 610 terminated by the distal tip 611 with cutting element 612. The first and second parts 610, 620 are foldable structures, such as flexible frame or mesh structures, each being deployable into a radially expanded shape.

The first part 610 hinged at its distal end to a stem 621 of the device 600 and opens to unfold at a proximal end. The second part 620 is hinged at a proximal end and opens to unfold at a distal end. The first and second parts 610, 620 have cooperating locking means 628, 629 adapted to connect the first and second parts 610, 620 in a fixed position with respect to each other. Advantageously, the cooperating locking means are adapted to fix the second part to the first part in a snap fit engagement with one or more snap positions. For example, locking means 629 may comprise snap lock tongues arranged at a proximal end of the second part 620. Cooperating locking means 628 may comprise snap fit ridges arranged on the stem 621 and are adapted for providing a snap fit engagement with the snap tongues when the second part is pushed onto the stem 621 by means of the pushing rod 510.

By way of example, FIG. 26 shows a detail of the device 600 with cooperating locking means 628, 629 connecting the proximal end of the second part 620 to the stem 621 in a snap fit engagement.

Like the pushing rod 510, the stem delivery tube 617 also extends in a proximal direction all the way to the proximal end of the endoscopic catheter 410 for manipulation by a user. A central lumen 627 extends axially through the entire stem delivery tube 617 and through the stem 621, all the way to the distal tip 611, and allows for the insertion of a guide wire 607, which also can be manipulated by a user at the proximal end of the endoscopic catheter 410. A distal portion of the guide wire 607 is also shown in FIG. 24.

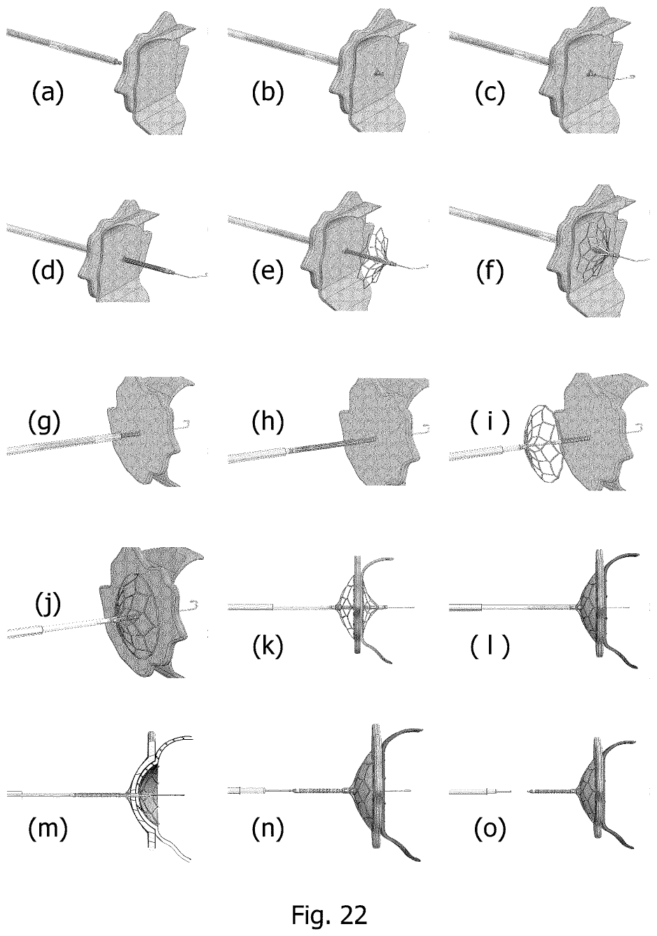

In FIG. 25, the gastroenteroanastomosis device 600 is seen in its deployed state in a cross-section taken in an axial plane passing through the center of the device and comprising the central axis A. The device 600 has a first part 610 connected to a second part 620 via a centrally arranged mechanical connection adapted for maintaining a fixed placement of the first and second parts 610, 620 with respect to each other. As mentioned above, the mechanical connection may be configured to comprise one or more pre-determined settings for the fixed placement, such as in a snap-fit engagement with one or more snap positions. The device 600 is introduced into a patient and placed at the anastomosis site using a minimally invasive intervention, where the two parts 610, 620 are introduced and placed at the gastroenteric target site, e.g. using a procedure with steps as further detailed in the example referring to FIG. 22 below.

The first part 610 is for placement on the enteric side, and is adapted to engage the tissue wall 99 of the small intestines at the target region from the inside of the small intestines. The second part 620 is for placement on the gastric side, and is adapted to engage the stomach wall 98 from the inside of the stomach. The first and second parts 610, 620 are connected to each other by a connection penetrating the engaged tissue wall portions in a central portion thereof, through an opening with a small diameter, and enclosed by circumferential engagement surfaces 615, 625 as further detailed in the following.

In the deployed state, the second part 620 is seated against the first part 610, the two parts engaging the two apposed tissue walls between them. When installing the device 600, the mutual position of the first and second parts 610, 620 is adjusted according to a desired pressure distribution applied to the engaged tissue walls.

Advantageously in some embodiments, the device 600 and/or an endoscopic system for the delivery and placement of the device 600 comprises a sensor element (not shown here) that provides an output indicative of an axial tension applied to pull the first and second parts 610, 620 towards each other. Collecting this output by a wired connection or by wireless communication allows to monitor and/or document the proper tensioning of the device during installation, and if the sensor element is arranged in the device 600 itself, even during the period of placement of the device in a patient for the formation of a gastroenteroanastomosis passage. For example, the sensor element may be adapted to communicate with an indicator device (not shown here) arranged at a proximal end of the endoscope. The indicator device may then provide user-perceptible output to the operator/user, thus allowing the operator/user to monitor and document the correct placement of the device. Furthermore, the indicator device, or a further data processing device (not shown here) may also collect the output from the sensor element for the purpose of monitoring, logging, and documenting correct placement and/or proper tensioning of the device.

Once the two parts 610, 620 are installed with proper tension forcing the first and second parts 610, 620 towards each other, their placement with respect to each other is typically fixed until the device eventually is removed in a second intervention, after formation of the gastroenteroanastomosis passage, as described above.

An axial direction of the device 600 is defined in its deployed state by a central axis A extending from a distal end 611 to a proximal end 622. When the device is in the collapsed state, the central axis A and the axial direction are also parallel to the direction of advancement for endoscopic insertion into a patient. When the device 600 is placed in the patient for forming a gastroenteroanastomosis passage, the axial direction corresponds to the direction of penetration of the tissue walls 98, 99. When installed, the axial direction of the device 600 is essentially perpendicular to the general direction of the tissue walls 98, 99 at the site of placement. Transverse directions of the device 600 are defined as parallel to a plane that is perpendicular to the axial direction, i.e. perpendicular to the central axis A. Radial directions are transverse directions intersecting the central axis A. The terms “central”, “circumferential”, “outward”, or “peripherally outward” are understood as seen in transverse directions of the device 600. The term “central” means typically close to the stem 621 connecting the first and second parts 610, 620 with each other, or comparatively closer to the stem 621 as compared to a location on the device described as peripherally outward. In a gastroenteroanastomosis device, such as the one shown in FIG. 25 an outward direction thus points in a transverse direction, typically radially, away from the central axis A and the centrally located stem 621.

The first part 610 of device 600 has radially extending elements 613, here provided as a foldable frame or mesh structure originating at the distal end 611, which in the deployed state is shaped to curve outwardly and away from the central axis A, thereby forming a convex surface facing towards the enteric wall 99 and towards a cooperating concave surface formed by the second part 620. The radially extending elements 613 are collapsible to fit within a radial circumference of a given channel allowing for the minimally invasive delivery of the first part 610, e.g. through an instrument channel of an endoscope as illustrated in FIG. 23. In the embodiment of FIG. 25, also the second part 620 of device 600 has radially extending elements 623, which may be provided as a foldable frame or mesh structure, and which is hinged at the proximal end having locking elements 629. In the deployed state, the second part is shaped to curve outwardly and away from the central axis A like, thereby forming a concave surface facing towards the gastric wall 98 and towards the cooperating convex surface formed by the first part 610. Like the elements 613 of the first part, the radially extending elements 623 are collapsible to fit within a radial circumference of a given channel allowing for the minimally invasive delivery of the second part 620, e.g. through an instrument channel of an endoscope as illustrated in FIG. 23. The frame or mesh structures may be made of any suitable material allowing for assuming a collapsed state and an extended state. For example, a frame or mesh structure may be produced as shapes that are mechanically biased towards an extended state, which may be held together in the collapsed state by a sleeve, and which may be released to their extended state by removing the sleeve upon introduction and placement. The sleeve may be of any suitable construction, such as a foil wrapping that can be broken and removed, or a wall of the catheter 410 used for delivery of the first part 610. The frame or mesh structure may also comprise a flexible braided wire mesh that upon axial compression assumes an outwardly bulging shape biased towards a generally convex/concave, i.e. cup-like shape. For example, an axial end of a braided mesh tube aligned in the axial direction A may be pulled inward and towards the other and to partially invert the tube into itself, such that an equatorial portion between the two ends bulges outward to form a peripheral rim of an open cup-shape. The frame or mesh structure may be made of any suitable biocompatible material. The frame or mesh structure may also comprise memory materials, such as nitinol, configured to assume a collapsed state, and upon activation an extended state, where activation is after introduction and placement of the first part 610 in its enteric location.

The stem delivery tube 617 extends from the proximal end 622 of the device 600 along the central axis A of the device 600 to an opposite end of the stem delivery tube 617 outside the patient. The distal portion of the stem delivery tube 617 is detachably connected to the stem 621 by releasable coupling means 618, 619. By way of example, FIG. 27 shows a detail of the device 600 with cooperating coupling means 618, 619 for releasably connecting the distal end of the stem delivery tube 617 to the proximal end 622 of the stem 621. FIG. 27 (a) shows the stem delivery tube 617 connected to the stem 621, and FIG. 27 (b) shows the stem delivery tube 617 disconnected from the stem 621. The releasable coupling means 618, 619 may be any suitable means, such as a form-fit connection as indicated in FIG. 27, a threaded connection, or similar releasable connections. FIG. 25 illustrates a situation during placement of the gastroenteroanastomosis device, where the pushing rod 510 pushes the proximal end of the unfolded second part 620 towards and into fixed connection with the first part 610, and where the stem delivery tube 617 is still connected to the device 600 to exert an axial pulling force pulling the first part 610 towards the second part 620 against the axial pushing force from the pushing rod 510. Furthermore, the endoscopic catheter 410 is already retracted. Also seen in FIG. 25 is the guide wire 607 passing through the inner channel 627 of the stem delivery tube 617 and the hollow stem 621, and projecting from the tip 611 at the distal end of the device into the lumen of the small intestines. Typically, the guide wire may during installation extend at least 5 cm, at least 10 cm, at least 20 cm, and up to 50 cm or even up to 1 m into the small intestines so as to secure the alignment of the target portions of the gastric and enteric walls during the installation procedure. It may be noted that in some embodiments, a guide wire that extends all the way from a manipulation end into the small intestines may be omitted, and a continuous inner channel in the stem delivery tube and stem may not be required. In some embodiments, the stem delivery tube may be a flexible stem delivery rod, and/or the stem may be provided without an inner channel 627.

Once the second part 620 is in place, it may be locked in fixed engagement with respect to the first element 610 using the locking elements 628, 629. The stem 621 thus cooperates with the proximal end of the first part 610 to link the first and second parts 610, 620 together. The stem 121 is thus part of the connection connecting the first and second parts 610, 620.

As mentioned above, in the deployed state the first part 610 presents a convex surface towards a cooperating concave surface of the second part. The convex surface of the first part 610 facing towards the tissue walls comprises a first engagement region 615 for seating a cooperating second engagement region 625 on the cooperating concave surface of the second part. The convex surface of the first part 610 further comprises a first peripheral region 616 located peripherally outward of the first engagement region 615, and facing a cooperating second peripheral region 626 on the cooperating concave surface of the second part 620.

The concave surface formed by the frame structure 623 of the second part 620 cooperates with the convex surface of the first part 610 to guide the two parts 610, 620 into proper alignment with respect to each other, such that the first engagement surface 615 is aligned vis-a-vis the second engagement surface 625, and the first peripheral surface 616 is aligned vis-a-vis the second peripheral surface 626. The cooperation of a concave surface on one of the first and second parts 610, 620 with a convex surface on the other one of the first and second parts 610, 620 facilitates self-alignment of the first and second parts 610, 620 with respect to each other. This ensures proper alignment of the engagement surfaces 615, 625 and of the peripheral surfaces 616, 626 with respect to each other-both when seating the second part 620 against the first part 610 during placement of the device 600, and for properly keeping the parts 610, 620 of the device 600 in place throughout the period of forming the gastroenteroanastomosis. Furthermore, the number of degrees of freedom to be controlled by the practitioner for installing the device 600 is reduced. As a result, an efficient installation, and a safe device for reliably forming a gastroenteroanastomosis passage is achieved.

The convex surface of the first part 610 has a circumferential first engagement region 615 adapted for seating a cooperating circumferential second engagement region 625 of the second part 620. When the device 600 is installed at the anastomosis site, the first and second engagement regions 615 and 625 engage the tissue walls with ischemic pressure at least along a circumferentially closed contour, thereby causing necrosis of the tissue, eventually opening a passage defined by the circumferential contour of the cooperating engagement surfaces 615, 625. In other words, upon installation, the cooperating first and second engagement regions of the first and second part are placed with respect to each other so as to define an ischemic pressure region with a circumferential contour of the desired passage opening. Furthermore, the cooperating first and second parts 610, 620 are flexible and/or tensioned so as to maintain an ischemic pressure throughout the period of installation of the device, and to accommodate any decrease in tissue thickness during the necrosis process.

Peripherally adjacent thereto in a radially outward direction, and enclosing the engagement region, a first peripheral region 616 of the first part 610 cooperates with a second peripheral region 626 of the second part 620 to form a region of gradually diverging surfaces. In this peripheral region, the distance between the cooperating surfaces 616, 626 of the first and second parts 610, 620 gradually increases in a radially outward direction, from a distance between the first and second engagement regions 615, 625, which is adapted for ischemic engagement, to a distance equal to, or exceeding, the sum of thicknesses of the uncompressed tissue walls. When the device 600 is installed at the anastomosis site, the gradually diverging surfaces of the first and second peripheral regions 616, 626 gradually release the pressure applied to the tissue walls from an ischemic pressure between the cooperating engagement surfaces 615, 625, to no pressure when the distance between the first and second peripheral surfaces 616, 626 exceeds the combined thickness of the uncompressed tissue walls. The cooperating first and second peripheral regions 616, 626 of the first and second part are thus adapted to keep the tissue walls in sealed contact with each other in a region peripherally enclosing the ischemic pressure region. The peripheral seal is provided right from the beginning, upon introduction and placement in the patient, and to provide a region of non-ischemic pressure, so that the tissue walls can heal together in this region, while a passage forms by necrosis in the engagement region enclosed by the peripheral seal. Thereby, a particularly safe way of forming the gastroenteroanastomosis passage is provided, where leaks of gastric fluids into the abdominal cavity are suppressed right from the beginning.

As mentioned above, the gradually diverging surfaces 616, 626 in the peripheral region provide a gradual pressure zone ensuring that the pressure applied to the tissue walls gradually decreases from an ischemic pressure in the engagement region between the first and second engagement surfaces 615, 625, to safely reach a non-ischemic regime where the tissue walls can heal together. The gradual pressure zone thus ensures a particularly safe and reliable formation of the peripheral seal for the gastroenteroanastomosis. Furthermore, the degree of divergence of the cooperating first and second peripheral surface regions 616, 626 may be determined, thus determining the width of the gradual pressure zone formed between these peripheral surface regions 616, 626, as seen in a transverse direction along the surface regions 616, 626. By determining the width of the gradual pressure zone where a non-ischemic pressure is applied, the width of the safe sealing seam surrounding the opening of the gastroenteroanastomosis passage is determined. Typically, the width of the gradual pressure zone is between 2 mm and 2 cm; advantageously, the width of the gradual pressure zone is between 3 mm and 15 mm; and further advantageously, the width of the gradual pressure zone is between 4 mm and 1 cm. The gradual pressure zone thus provides a reliable method for configuring the device to provide a safe and reliably sealed gastroenteroanastomosis.

The engagement region formed by the cooperating first and second engagement surfaces 615, 625 also encloses the centrally arranged mechanical connection connecting the first and second parts 610, 620, thereby sealing the penetration during the necrotic formation of the passage. Furthermore, since the peripheral regions 616, 626 enclose the engagement regions 615, 625, they also enclose the centrally arranged penetration for the connection. The small initial penetration produced by the centrally arranged mechanical connection is thus also sealed off by the non-ischemic peripheral seal provided by the cooperating first and second peripheral regions 616, 626. The risk of leakage of gastric fluids into the abdominal cavity is therefore minimized.

The embodiment shown in FIGS. 23-27 has rotational symmetry around the central axis A of the device 600, thereby being adapted to form an essentially circular gastroenteroanastomosis passage. A circular symmetry has the advantage that the pressure distribution is essentially invariant in a circumferential direction along the contours defining the engagement and peripheral regions 615, 625, 616, 626. Thereby a well-controlled and predictable performance is achieved for the device 600 having a first part 610 with a foldable wire frame 613 and a cooperating second part with an inflatable body 123. However, other cooperating shapes of the foldable frame structures 613, 623 are generally conceivable, such as a shape that is elongated in one transverse direction (e.g. longitudinal) as compared to another transverse direction (e.g. cross-wise), thus providing correspondingly elongated engagement and peripheral surfaces and resulting in an elongated shape of the gastroenteroanastomosis passage formed by such an elongated device. Furthermore, the elements composing the foldable frame structures may be shaped and dimensioned to minimize transverse dimensions of the first and/or second parts in the collapsed state.

### EXAMPLES

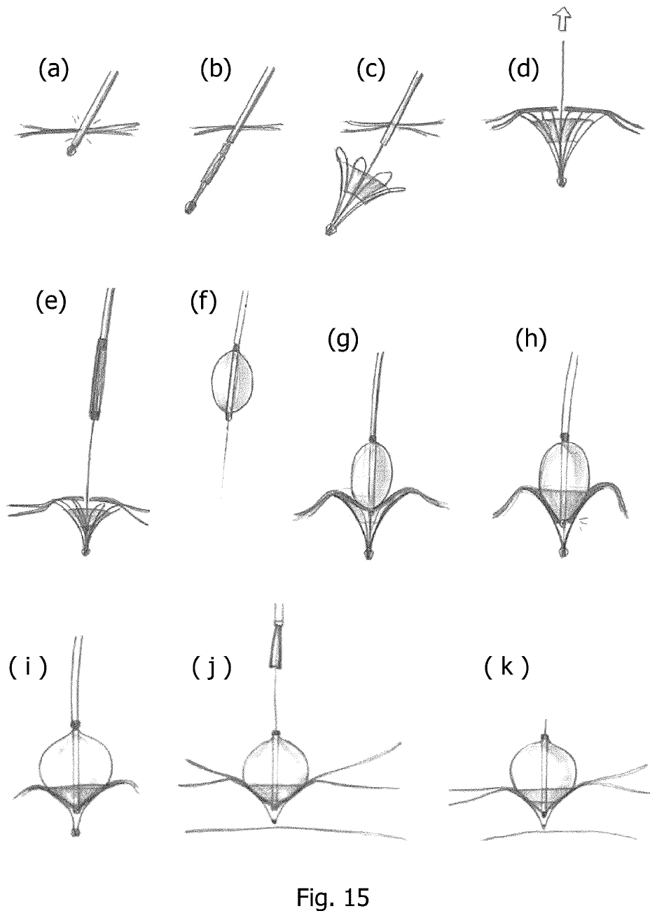

Referring to FIGS. 15-22 in the following, examples are given for using embodiments of the gastroenteroanastomosis device for forming a passage from the stomach to a target portion of the small intestines in a gastric bypass procedure, such as described above.

Referring to FIG. 15, example 1 describes steps (a)-(k) of placing a gastroenteroanastomosis device 100 according to the embodiment of FIGS. 4-6 during a first intervention of a gastric bypass procedure, wherein the references to elements of the device 100 given in the following correspond to the references in FIGS. 4-6. By way of example, the gastric bypass procedure may be performed as described with reference to FIG. 3 above, wherein FIG. 3(e) represents placement of the gastroenteroanastomosis device. Turning again to FIG. 15, a catheter 410 encasing the collapsed first part 110 of device 100 is endoscopically introduced into the stomach, and advanced to the apposed tissue walls. The cutting element 112 on the tip 111 of the first part 110 protrudes from the end of the catheter 410 and is activated to cut through the tissue and the catheter 410 is further advanced to penetrate the apposed tissue walls from the stomach towards the target region of the small intestines (step (a)). The first part 110 is then pushed forward and released from the catheter 410 to unfold into the expanded state (steps (b) and (c)). The catheter 410 is removed, leaving behind the first part 110 attached to a guidewire 117 extending from the distal tip all the way to the opposite end outside the patient. By means of the guide wire 117, the first part is then pulled against the enteric side of the apposed tissue walls (step (d)). Using a pushing tube 510, the second part 120 is descended along the guidewire 117 in a collapsed state, approaching the apposed tissue walls that are held up by the first part from the gastric side (step (e)). Before contacting the tissue walls, the inflatable body 123 is inflated to a first pressure to unfold it (step (f)). The second part 120 with the unfolded inflatable body 123 is pushed forward to catch the apposed tissue walls (step (g)). The second part 120 is pushed further, to engage the apposed tissue walls between cooperating surface portions on the inflatable body 123 of the second part 120 and on the wire frame 113 of the first part 110 (step (h)). The interaction between the concavely shaped receiving surface on the first part and the cooperating convexly shaped outer surface of the second part assists in the easy and reliable alignment, essentially self-aligning the two parts 110, 120 of the gastroenteroanastomosis device 100 as they are forced towards each other. The second part is locked in place by fixing it to the distal portion of the guidewire 117 connecting the first and second parts 110, 120 to each other. The inflatable body is further pressurized to an operating pressure, configured to define circumferential engagement regions 115, 125 engaging the apposed tissue walls with a pressure in an ischemic regime, and at the same time to define diverging peripheral surface regions 116, 126 located adjacent thereto and peripherally enclosing the engagement regions 115, 125 in a peripherally outward direction (step (i)). As discussed when describing various embodiments of the gastroenteroanastomosis device disclosed herein, the peripheral regions 116, 126 cooperate to gradually reduce the pressure applied to the apposed tissue walls from the ischemic regime, at the engagement regions 115, 125, to no pressure, at the periphery of the device. The cooperating peripheral surface regions are thus configured to apply a non-ischemic pressure to the apposed tissue walls to form a seal of a predetermined width as seen in a radial direction from the engagement regions 115, 125 to the periphery, corresponding to the width of the cooperating peripheral regions 116, 126 in this direction. After placement and proper pressurization, the guidewire is cut close to the installed device 100 (step (j)), and all endoscopic instrumentation is removed only leaving the device 100 behind for forming the gastroenteroanastomosis passage (step (k)). While the device 100 is installed in the apposed tissue walls, i.e. during the process of forming the gastroenteroanastomosis passage, there is no opening yet, which directly connects the stomach to the target region of the small intestines to bypass the pylorus and the duodenum. The passage is only opened by removing the device in a subsequent second intervention, after the gastroenteroanastomosis is completed.

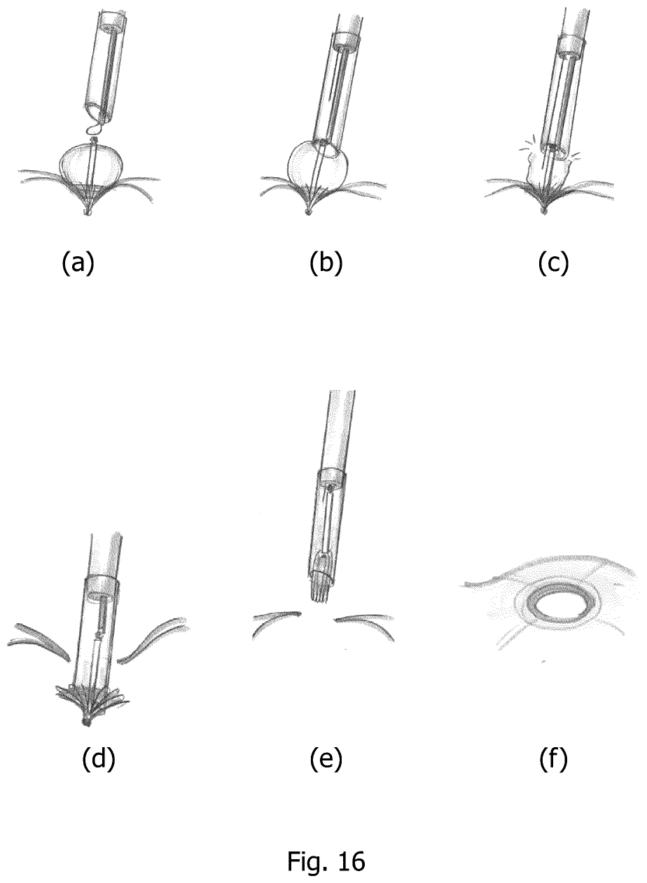

Referring to FIG. 16, example 2 describes steps (a)-(f) of retrieving a gastroenteroanastomosis device 100 according to the embodiment of FIGS. 4-6 during a second intervention of a gastric bypass procedure. As in example 1, any reference numbers to elements of the device 100 relate to the reference numbers shown in FIGS. 4-6. In the second intervention, the passage is opened by removing the device 100 after gastroenteroanastomosis is complete, e.g. as described above with reference to FIG. 3(g) and FIG. 3(h). Turning again to FIG. 16, a catheter carrying an endoscopic instrument is introduced into the stomach and advanced to the proximal end of the device 100 (step (a)). The endoscopic instrument is adapted for coupling to the coupling element 124 at the proximal end of the device 100, such as a snare catching a groove 124 in a flange at the proximal end of the device 100. The endoscopic instrument catches and holds the device 100 at the coupling element 124, and the inflatable body is punctured to collapse, thereby removing the pressure applied to the tissue walls (step (c)). The end of the catheter carries a collar that is now further advanced into the enteric side while pulling back the endoscopic instrument, to push against the wire frame of the first part, thereby capturing any necrotic tissue (step (d)). Further pulling the first part into the collar forces the wire frame to flip around, which then inverts and collapses like an umbrella in the storm, whereby it can be retrieved in a gastric direction, thereby opening the gastroenteroanastomosis passage (steps (e) and (f)). In some embodiments, to facilitate the flipping around, the collar may have an angled cut-off-shape adapted to force one side of the wire structure to fold back before the rest of the wire structure follows. This is advantageous in an embodiment, where the membrane attached to the umbrella has a constant perimeter, and is difficult to rupture. This allows for deforming the wire frame structure while flipping it around, so as to maintain a constant perimeter during the flipping operation.

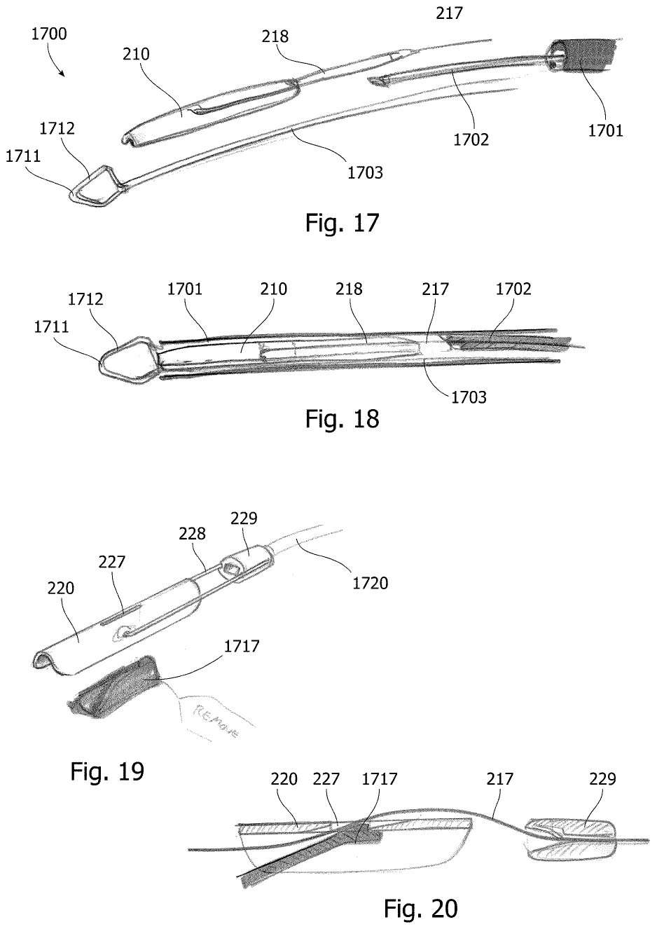

Referring to FIGS. 17-20, example 3 describes a kit 1700 for placing a gastroenteroanastomosis device according to the embodiment of FIGS. 7-9. The kit comprises a catheter casing 1701, a first pushrod manipulator 1702, a cutting device 1703 with a cutting element 1712 arranged at a distal tip 1711 thereof, and a first part 210 of an elongated bar-shaped gastroenteroanastomosis device 200. The first part 210 has centrally attached connection elements 218 allowing the bar-shaped body portion of the first part 210 to tilt about a cross-wise axis at the distal attachment points, between a collapsed state and an expanded state as described above with reference to FIGS. 7-9. At their proximal end, the connection elements 218 are combined and connected to a guidewire 217. As best seen in FIG. 18, the casing 1701 sheathes the first part 210 in its collapsed state, the first manipulator 1702, and the cutting device 1703. The guidewire 217 is threaded through a hollow core of the first manipulator 1702, which in an axial direction along a principal axis of the casing 1701 is arranged proximal of the first device 210. Advantageously, the cutting device 1703 is an electrical cutting device with an electrically heated cutting element 1712. The tip portion 1711 with the cutting element 1712 protrudes from the distal end of the casing 1701. A shaft of the cutting device 1703 passes by the first part 210 and the first manipulator 1702, and extends along the axial direction to the distal end of the casing 1701. The kit 1700 further comprises the second part 220 of the device 200, a threading device 1717, and a second pushrod manipulator 1720. The second part 220 has a trough shaped body portion with a central opening 227, stiff connection elements 228 with distal ends hinged to the body portion to allow for tilting the body around a cross-wise axis, and a locking element 229 attached to the proximal ends of the connection elements 228. The threading device 1717 is inserted in the second part for facilitating threading of the guidewire 217 through the central opening 227 of the body portion of the second part 220, as best seen in FIG. 20. The guidewire is further passed through the locking element 229, and through a hollow core of the second manipulator 1722, all the way to the proximal end of the second manipulator 1722.

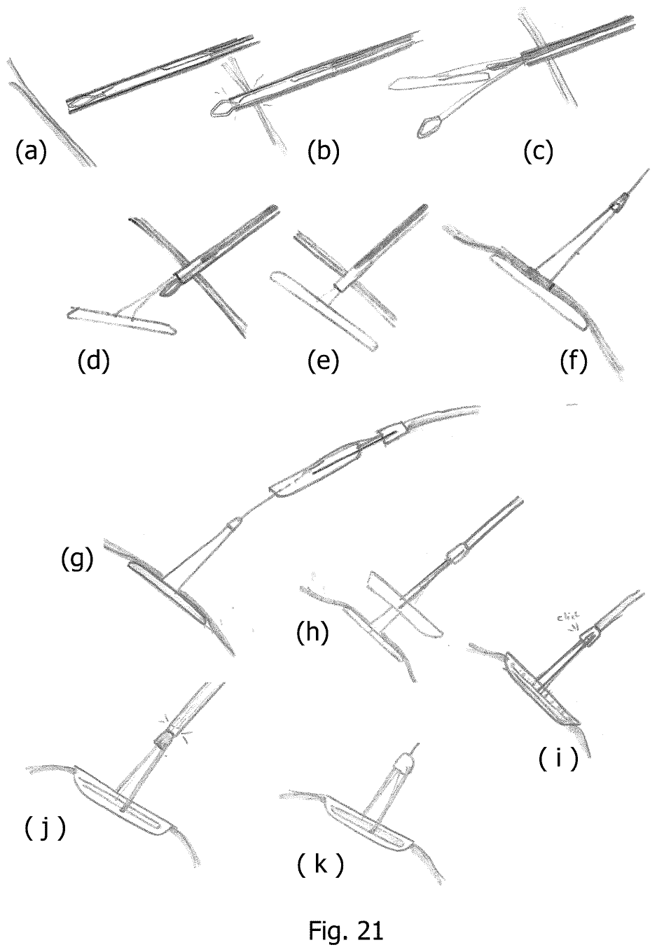

Now referring to FIG. 21, example 3 further describes steps (a)-(k) of placing a gastroenteroanastomosis device 200 according to the embodiment of FIGS. 7-9 during a first intervention of a gastric bypass procedure. References in the following to elements of the kit and devices used in these steps refer to those described above with respect to FIGS. 7-9, and FIGS. 17-20. By way of example, the gastric bypass procedure may be performed as described with reference to FIG. 3 above, wherein FIG. 3(e) represents placement of the gastroenteroanastomosis device. Turning again to FIG. 21, catheter 1701 holding the first manipulator 1702, the first part 210 of device 200, and the cutting device 1703, is introduced endoscopically and advanced to the apposed tissue walls (step (a)). The cutting device is deployed and activated to penetrate the apposed tissue walls from the gastric side and into the target region of the small intestines (step (b)). Using the first manipulator 1702, the first part 210 is pushed out of the catheter casing 1701, past the cutting device 1703, into the small intestine tube (step (c)). The first part 210 is tilted into its extended state, the cutting device 1703 is retracted and together with the casing 1701 and the second manipulator removed from the patient, leaving the first part 210 placed against the enteric side of the apposed tissue walls with the connection elements 218 penetrating into the stomach under tension of the guidewire 217 (steps (d)-(f)). Note that the direction of elongation of the first part 210 is aligned essentially along the longitudinal direction, parallel to the general direction of the small intestine tube in the target region. The second part 220 is then threaded onto the guidewire 217 as described above with reference to FIG. 20, and introduced along the guidewire in its collapsed state by means of the second manipulator 1720 (step (g)). The trough-shaped second part 220 is then tilted into its extended state and placed against the gastric side of the apposed tissue walls, with the connection elements 218 of the first device passing through the slit-shaped central opening 227 (steps (g)-(i)). The connection elements 218 of the first part 210 are attached to the body portion of the first part 210 in a longitudinal direction. The central slit-shaped opening 227 in the second part 220 is also aligned with the longitudinal direction. Thereby, the second part 220 is naturally guided towards a longitudinal orientation, parallel to the first part 210, as it approaches its placement against the gastric wall. The concave trough shape furthermore self-aligns with the convex bar shape of the first part as the two parts seat against each other to engage the apposed tissue walls between them. The second part is via stiff connection elements 228 connected to a locking element 229. The locking element is fixed to the guide wire and connection elements 218 at a desired location, thus forcing the second part 220 towards the first part 210 and fixing the parts with respect to each other (step (i)). The guidewire 217 is then cut and the instrumentation removed from the patient, leaving behind only the elongated gastroenteroanastomosis device 200 in longitudinal orientation to form the gastroenteroanastomosis passage (steps (j)-(k)). As also discussed above with respect to FIGS. 7-9, when placed in this manner, the device 200 is configured to define engagement regions 215, 225 engaging the apposed tissue walls with a pressure in an ischemic regime, and at the same time to define diverging peripheral surface regions 216, 226 located adjacent thereto and peripherally enclosing the engagement regions 215, 225 in a peripherally outward direction. As discussed above when describing the various embodiments of the gastroenteroanastomosis device, the peripheral regions 216, 226 cooperate to gradually reduce the pressure applied to the apposed tissue walls from the ischemic regime, at the engagement regions 215, 225, to no pressure, at the periphery of the device. The cooperating peripheral surface regions are thus configured to apply a non-ischemic pressure to the apposed tissue walls to form a seal of a predetermined width as seen in an outward direction from the engagement regions 215, 225 to the periphery, essentially corresponding to the width of the cooperating peripheral regions 216, 226 in this direction. Also note that while the device 200 is installed in the apposed tissue walls, i.e. during the process of forming the gastroenteroanastomosis passage, there is no opening yet, which directly connects the stomach to the target region of the small intestines to bypass the pylorus and the duodenum. The passage is only opened by removing the device in a subsequent second intervention, after the gastroenteroanastomosis is completed. For devices of the type of device 200 or device 300, this may be performed in an analogous manner as described in example 2 above with respect to a device of the type of device 100.

Referring to FIG. 22, example 4 describes steps (a)-(o) of placing a gastroenteroanastomosis device 600 according to the embodiment of FIGS. 23-27 during a first intervention of a gastric bypass procedure, wherein the references to elements of the device 600 given in the following correspond to the references in FIGS. 23-27. By way of example, the gastric bypass procedure may be performed as described with reference to FIG. 3 above, wherein FIG. 3(e) represents placement of the gastroenteroanastomosis device. Turning again to FIG. 22, a catheter 410 encasing the collapsed first and second parts 610, 620 of device 600 is endoscopically introduced into the stomach, and advanced to the apposed tissue walls (step (a)). The cutting element 612 on the tip 611 of the first part 610 protrudes from the end of the catheter 410 and is activated to cut through the tissue and the catheter 410 is further advanced to penetrate the apposed tissue walls from the stomach towards the target region of the small intestines (step (b)). The guidewire 607 is advanced through the channel 627 to extend into the small intestines in order to secure the alignment of the gastric and enteric target regions during the placement procedure (step (c)). The first part 610 is then pushed forward and released from the catheter 410 to unfold into the expanded state in the target region of the small intestines (steps (d) and (e)). The unfolded first part 610 is then pulled tight against the enteric wall 99, e.g. by pulling the stem delivery tube “backwards”, i.e. in a proximal direction (step (f)). The endoscopic catheter 410 can then be retracted to release and unfold the second part on the gastric side (steps (g)-(i)). Using pushing tube 510, the unfolded second part 620 is pushed “forward”, i.e. in a distal direction, towards and against the gastric side of the apposed tissue walls that are held up by the first part 610 (steps (j)-(k)). Pushing further advances the locking elements 629 on the second part 620 into engagement with the cooperating locking elements 628 on the stem 621. Pushing further also leads to an inversion of the unfolded first part 610 so as to form a generally convex/concave cup-like structure with a convex surface facing towards the concave surface of the second part 620 (steps (I)-(m)). The state of the device 600 seen in step (m) corresponds to the deployed device 600 as seen in FIG. 25. The device 600 now engages the apposed tissue walls between cooperating surface portions on the foldable structure 623 of the second part 620 and on the foldable structure 613 of the first part 610. The interaction between the concavely shaped receiving surface on the second part and the cooperating convexly shaped inverted surface of the first part assists in the easy and reliable alignment, essentially self-aligning the two parts 610, 620 of the gastroenteroanastomosis device 600 as they are forced towards each other. The second part is locked in place by fixing it to the proximal portion of the stem 621 connecting the first and second parts 610, 620 to each other with an axial tension forcing the two parts together. The axial tension is configured to define circumferential engagement regions 615, 625 engaging the apposed tissue walls with a pressure in an ischemic regime, and at the same time to define diverging peripheral surface regions 616, 626 located adjacent thereto and peripherally enclosing the engagement regions 615, 625 in a peripherally outward direction (step (n)). As discussed when describing various embodiments of the gastroenteroanastomosis device disclosed herein, the peripheral regions 616, 626 cooperate to gradually reduce the pressure applied to the apposed tissue walls from the ischemic regime, at the engagement regions 615, 625, to no pressure, at the periphery of the device 600. The cooperating peripheral surface regions are thus configured to apply a non-ischemic pressure to the apposed tissue walls to form a seal of a predetermined width as seen in a radial direction from the engagement regions 615, 625 to the periphery, corresponding to the width of the cooperating peripheral regions 616, 626 in this direction. After placement and proper tensioning of the first and second parts towards each other, the detachable coupling 618, 619 between the stem delivery tube 617 and the stem 621 is disconnected, and all endoscopic instrumentation is removed only leaving the device 600 behind for forming the gastroenteroanastomosis passage (step (o)). While the device 600 is installed in the apposed tissue walls, i.e. during the process of forming the gastroenteroanastomosis passage, there is no opening yet, which directly connects the stomach to the target region of the small intestines to bypass the pylorus and the duodenum. More generally, the devices of the present disclosure are not conceived to provide a transluminal shunt from the stomach to the small intestines while they are installed. The passage is only opened by removing the device 600 in a subsequent second intervention, after the gastroenteroanastomosis is completed. Removal of the device 600 may be performed in an analogue manner as described above with respect to FIG. 16, steps (a), and (d)-(f).

1. A gastroenteroanastomosis device for use in a minimally invasive procedure for forming a passage across apposed tissue walls from the stomach to a target portion of the small intestines in a patient; the device being transformable from a collapsed state configured for minimally invasive delivery of the device to a gastro-enteric deployment site in the apposed tissue walls to a deployed state configured for deployment at the gastro-enteric deployment site; the device comprising when seen in the deployed state:
a first part adapted for enteric placement;
a second part adapted for gastric placement; and
a centrally arranged connection forcing the first and second parts towards each other in an axial direction;
wherein the first part comprises a first engagement region, wherein the second part comprises a second engagement region facing towards the first engagement region, and wherein the first and second engagement regions are adapted to engage the apposed tissue walls there between to apply an ischemic pressure;
wherein the first part further comprises a first peripheral region enclosing the first engagement region in a peripherally outward direction, wherein the second part further comprises a second peripheral region enclosing the second engagement region in a peripherally outward direction and facing towards the first peripheral portion, and wherein the first and second peripheral regions are adapted to contact the apposed tissue walls there between to apply a non-ischemic pressure;
wherein the first and second peripheral regions diverge from each other, whereby the first and second peripheral regions cooperate to gradually reduce, in an outward going direction from the engagement region to a periphery of the device, a pressure applied to the apposed tissue walls from ischemic pressure at the engagement region to no pressure at the periphery, passing through non-ischemic pressure.

2. Device according to claim 1, wherein the distance between the cooperating peripheral regions increases monotonically, or strictly monotonically, from an inner distance at the peripherally outer border of the engagement regions to an outer distance at the peripherally outer border of the peripheral regions.

3. Device according to claim 1, wherein a first one of the first and second cooperating parts has a concave shape facing towards the other one of the first and second parts.

4. Device according to claim 3, wherein the other one of the first and second parts has a convex shape facing towards the concave first one of the first and second parts.

5. Device according to claim 1, wherein a peripheral contour of the cooperating engagement regions of the first and second parts has a circular shape.

6. Device according to claim 1, wherein a peripheral contour of the cooperating engagement regions of the first and second parts has an elongated shape.

7. Device according to claim 1, wherein the first part and/or the second part comprises a collapsible frame structure having a collapsed state adapted for minimally invasive delivery and an expanded state adapted for deployment at the gastro-enteric deployment site.

8. Device according to claim 7, wherein the first part and/or the second part, as applicable, further comprises a foldable membrane attached to the collapsible frame structure, wherein the membrane is folded together when the collapsible frame structure is in the collapsed state, and wherein the membrane is unfolded to present a cooperating surface comprising engagement and peripheral regions, when the collapsible frame structure is in the expanded state.

9. Device according to claim 1, wherein a collapsible structure of the first part and/or the second part is biased towards an expanded state.

10. Device according to claim 1, wherein a tip portion of the first part comprises a cutting device, such as an electrically heated cutting wire.

11. Device according to claim 1, further comprising a sensor element adapted to provide an output indicative of a highest compression force applied between the first part and the second part.

12. Device according to claim 1, further comprising a sensor element adapted to provide a sensor output indicative of an axial tension forcing the first part and the second part towards each other.

13. Device according to claim 11, wherein the sensor element is arranged in the mechanical connection between the first part and the second part.

14. Device according to claim 11, wherein the device further comprises an indicator device in communication with the sensor element, the indicator device being adapted to produce a user-perceptible output corresponding to the sensor output.

15. Device according to claim 14, wherein the user-perceptible output is one or more of a visual output, an audible output, a haptic output, and a tactile output.

16. Device according to claim 14, wherein the indicator device communicates with the sensor through one or more of a wired connection and a wireless connection.

17. Endoscopic system for use in a minimally invasive gastroenteroanastomosis procedure for forming a passage across apposed tissue walls from the stomach to a target portion of the small intestines in a patient; the system comprising:
a gastroenteroanastomosis device according to claim 1;
an endoscope adapted for insertion into the stomach at a target site for forming a gastroenteroanastomosis passage, the endoscope comprising an instrument channel extending from a proximal end to a distal end of the endoscope;
an endoscopic catheter adapted for the delivery and placement of the gastroenteroanastomosis device through the instrument channel of the endoscope.

18. System according to claim 17, wherein the system further comprises a sensor element adapted to provide a sensor output indicative of an axial tension forcing the first part and the second part towards each other.

19. System according to claim 18, wherein the sensor element is arranged at a proximal end of the endoscope so as to determine an axial tension between a first control element for the manipulation of the first part and a second control element for the manipulation of the second part.

20. System according to claim 17, wherein the system further comprises an indicator device in communication with the sensor element, the indicator device being adapted to produce a user-perceptible output corresponding to the sensor output.

21. System according to claim 20, wherein the user-perceptible output is one or more of a visual output, an audible output, a haptic output, and a tactile output.

22. System according to claim 20, wherein the indicator device communicates with the sensor through one or more of a wired connection and a wireless connection, such as Bluetooth, BLE, or the like.

23. System according to claim 20, wherein the indicator device is arranged at a user end of the endoscope.
